SEQUENCE LISTING (12 sequences)

SEQ ID NO: 1 - protein, 27 aa, Artificial Sequence
  E7-BMP2
EEEEEEEKIPKASSVPTELSAISTLYL

SEQ ID NO: 2 - DNA, 20 bp, Artificial Sequence
  Runx2 forward primer
ccaacttcctgtgctccgtg

SEQ ID NO: 3 - DNA, 21 bp, Artificial Sequence
  Runx2 reverse primer
gtgaaactcttgcctcgtccg

SEQ ID NO: 4 - DNA, 22 bp, Artificial Sequence
  Alp forward primer
cggccatcctatatggtaacgg

SEQ ID NO: 5 - DNA, 21 bp, Artificial Sequence
  Alp reverse primer
caggaggcatacgccatcaca

SEQ ID NO: 6 - DNA, 21 bp, Artificial Sequence
  OCN forward primer
gaccctctctctgctcactct

SEQ ID NO: 7 - DNA, 22 bp, Artificial Sequence
  OCN reverse primer
gaccttactgccctcctgcttg

SEQ ID NO: 8 - DNA, 20 bp, Artificial Sequence
  BSP forward primer
aggtcgtcaacggcaccagc

SEQ ID NO: 9 - DNA, 20 bp, Artificial Sequence
  BSP reverse primer
ccataggcttcgggtggcgg

SEQ ID NO: 10 - DNA, 18 bp, Artificial Sequence
  GAPDH forward primer
gcaagttcaacggcacag

SEQ ID NO: 11 - DNA, 19 bp, Artificial Sequence
  GAPDH reverse primer
cgccagtagactccacgac

SEQ ID NO: 12 - protein, 20 aa, Artificial Sequence
  BMP-2 peptide
KIPKASSVPTELSAISTLYL

(19) United States
(12) Patent Application Publication
(10) Pub. No.: US 2023/0390456 A1
(43) Pub. Date: Dec. 7, 2023

(54) SURFACE MODIFIED SCAFFOLDS AND METHODS OF USE THEREOF

(71) Applicant: Board of Regents of the University of Nebraska, Lincoln, NE (US)

(72) Inventor: Jingwei XIE, Omaha, NE (US)

(21) Appl. No.: 18/026,502

(22) PCT Filed: Oct. 25, 2021
(86) PCT No.: PCT/US21/56435

§ 371 Date: Mar. 15, 2023

Related U.S. Application Data
(60) Provisional application No. 63/104,689, filed on Oct. 23, 2020.

(51) Int. Cl. A61L 27/34; A61L 27/56; A61L 27/18; A61L 27/54

(52) U.S. Cl. CPC A61L 27/34; A61L 27/56; A61L 27/18; A61L 27/54; A61L 2400/12; A61L 2400/18

(57) ABSTRACT

Surface modified scaffolds are provided as well as methods of use thereof and methods of making.

This application claims priority under 35 U.S.C. § 119(e) to U.S. Provisional Patent Application No. 63/104,689, filed Oct. 23, 2020. The foregoing application is incorporated by reference herein.

This invention was made with government support under Grant No. R21 DE027516 awarded by the National Institutes of Health. The government has certain rights in the invention.

### FIELD OF THE INVENTION

This application relates to the fields of surface modified scaffolds. More specifically, this invention provides surface modified scaffolds, methods of synthesizing, and methods of use thereof.

### BACKGROUND OF THE INVENTION

Several publications and patent documents are cited throughout the specification in order to describe the state of the art to which this invention pertains. Each of these citations is incorporated herein by reference as though set forth in full.

Tissue dysfunction caused by trauma, aging, and a myriad of diseases such as cancer, diabetes, and osteoarthritis have led to a serious decline in human life quality (Fisher, et al. (2013) Tissue Eng. Part B, 19:1; Leijten, et al. (2016) Small 12:2130; Shin, et al. (2016) Small 12:3677). The human body has the ability to restore damaged tissues to some extent. However, many factors influence the efficacy of regeneration, including the tissue type, demand for growth hormones, and critical-sized defects which cannot heal spontaneously without secondary interventions (Jammalamadaka, et al. (2018) J. Funct. Biomater., 9:22; Sanders, et al. (2014) Trauma 28:632). Autografts and allografts, the gold standard therapies in critical-sized tissue defect healing, suffer from several drawbacks including limited resources, size mismatch, immune rejection, and disease transmission (Heest, et al. (1999) Lancet 353:S28). Regenerative medicine, an interdisciplinary field that applies engineering and life science principles to promote regeneration, can potentially restore diseased and injured tissues and organs (Mao, et al. (2015) PNAS 112:14452). The use of functional biomaterials or scaffolds is a commonly used strategy of regenerative medicine. Conventional methods for fabricating scaffolds include solvent-casting, particulate-leaching, phase separation, melt-molding, and emulsion freeze-drying (Sachlos, et al. (2003) Eur. Cells Mater., 5:29; Wust, et al. (2011) J. Funct. Biomater., 2:119). Those approaches are often inadequate for generating scaffolds with precise pore size, specific pore geometry, high interconnectivity level, good mechanical strength, and homogeneous distribution of cells (Sachlos, et al. (2003) Eur. Cells Mater., 5:29; Wust, et al. (2011) J. Funct. Biomater., 2:119). Accordingly, new methods for the fabrication of scaffolds are needed.

### SUMMARY OF THE INVENTION

In accordance with the instant invention, surface modified scaffolds are provided. Methods of making the surface modified scaffolds are also provided.

In accordance with an aspect of the instant invention, methods of synthesizing a surface modified scaffold are provided. In certain embodiments, the method comprises coating the surface of a scaffold with a coating material and nanofiber segments, and crosslinking the coating or coating material on the surface of the scaffold. The method may further comprise plasma treating the scaffold prior to coating the surface of the scaffold. In certain embodiments, the crosslinking comprises contacting the coated scaffold with a chemical crosslinker such as glutaraldehyde. The methods of the instant invention may further comprise synthesizing the scaffold, such as by 3D printing, prior to coating. In certain embodiments, the nanofiber segments are electrospun nanofiber segments and/or have a length less than about 50 μm. In certain embodiments, the method further comprises adding an agent, cell, or tissue to the coating of the surface modified scaffold, either before or after the crosslinking. In certain embodiments, the coating material comprises gelatin. In certain embodiments, the scaffold comprises microfibers comprising polycaprolactone.

In accordance with another aspect of the instant invention, surface modified scaffolds are provided. In certain embodiments, the surface modified scaffold are synthesized a method of the instant invention. In certain embodiments, the surface modified scaffold comprises a) a plasma treated scaffold and b) a crosslinked coating comprising a coating material and nanofiber segments. In certain embodiments, the scaffold is a 3D printed scaffold. In certain embodiments, the nanofiber segments are electrospun nanofiber segments and/or have a length less than about 50 μm. In certain embodiments, coating material comprises gelatin. In certain embodiments, the scaffold comprises microfibers comprising polycaprolactone. In certain embodiments, the surface modified scaffold further comprises an agent, cells, and/or tissues in the coating. Compositions comprising the surface modified scaffold and a carrier are also encompassed by the instant invention.

In accordance with another aspect of the instant invention, methods of using the surface modified scaffolds are provided. For example, the surface modified scaffolds may be used to enhance wound healing, build tissue constructs, promote tissue regeneration (e.g., bone and/or cartilage regeneration), reduce, inhibit, prevent, and/or eliminate infection, local delivery of drugs, and/or inhibit bleeding.

### BRIEF DESCRIPTION OF THE DRAWINGS

FIG. 1 provides a schematic illustrating procedures for fabrication of PCL scaffold-PCG, PCL scaffold-BG, and PCL scaffold-BG-BMP2. 3D printed PCL scaffolds were first treated by plasma. For PCL scaffold-PCG, the PCL scaffolds were immersed in the gelatin and PCG short nanofiber solution for 1 hour. For PCL scaffold-BG, plasma-treated PCL scaffolds were immersed in gelatin solution for 20 minutes and then transferred into 5 or 10 mg/mL BG short nanofiber suspension in the acetic buffer (AcB). After 1 hour BG short nanofiber coating, the scaffolds were immersed in gelatin solution for 20 minutes. Glutaraldehyde vapor from ethanol solution was used to cross-link gelatin for 36 hours. PCL scaffold-BG was further modified by bone morphogenetic protein 2 (BMP2) mimicking peptide via soaking the scaffold in 300 μg/mL BMP2 peptide tris-buffered saline (TBS) solution for 24 hours. PCL scaffold-PCG: PLGA-collagen-gelatin short nanofibers coated 3D printed PCL scaffolds. PCL scaffold-BG: bioactive glass short nanofibers coated 3D printed PCL scaffolds. PCL scaffold-BG-BMP2: bioactive glass short nanofibers coated 3D printed PCL scaffolds conjugated with E7-BMP2 peptides. PCL scaffold-G: gelatin coated 3D printed PCL scaffolds. PCG: PLGA-collagen-gelatin. BG: bioactive glass.

FIG. 2A provides scanning electron microscopy (SEM) images of PCL scaffold, PCL scaffold-PCG (L: PCL scaffold-PCG10 and H: PCL scaffold-PCG20), and PCL scaffold-BG (L: PCL scaffold-BG5 and H: PCL scaffold-BG10). L and H represent low and high suspension concentrations of SFs, respectively. Scale bar in the insets=10 μm. FIG. 2B provides a representative image of PCL scaffold-BG5 acquired via microcomputed tomography (micro-CT) analysis. FIG. 2C provides a graph of compression stress profiles of various scaffolds as the deformation increases. Top line: PCL scaffold; middle line: PCL scaffold-PCG; bottom line: PCL scaffold-BG. FIG. 2D provides SEM images of PCL mesh, PCL mesh-PCG (L: PCL mesh-PCG10 and H: PCL mesh-PCG20), and PCL mesh-BG (L: PCL mesh-BG5 and H: PCL mesh-BG10). L and H represent low and high suspension concentrations of SFs, respectively.

FIG. 3A provides confocal laser scanning microscopy (CLSM) images of rat bone marrow-derived stem cells (rBMSCs) adhesion and proliferation on PCL scaffold, PCL scaffold-PCG20 and PCL scaffold-BG5 after 12 hours and 7 days of culture in the proliferating medium. Scale bar=200 μm. Scale bar in the insets=50 μm. FIG. 3B provides graphs of corresponding cell numbers on various scaffolds after incubation for 12 hours and 7 days. *p<0.05; n=3 per group. FIG. 3C provides CLSM images of rBMSCs adhesion and proliferation on PCL scaffold, PCL scaffold-PCG10 and PCL scaffold-BG10 after 12 hours and 7 days of culture in the proliferating medium. Scale bar=200 μm. Scale bar in the inset=50 μm. FIG. 3D provides graphs of corresponding cell numbers on various scaffolds over short and long culture periods. *p<0.05; n=3 per group.

FIG. 4 provides SEM images of rBMSCs on PCL scaffold, PCL scaffold-PCG20, and PCL scaffold-BG5 after 12 hours and 7 days of culture in the proliferating medium. Scale bar =20 μm. White arrows indicate filopodia.

FIG. 5A provides graphs of MC3T3 cell numbers on PCL scaffold, PCL scaffold-PCG10, PCL scaffold-PCG20, PCL scaffold-BG5, and PCL scaffold-BG10 after culture for 2 hours, 4 hours, 12 hours, and 4 days. *p<0.05; n =3 per group. FIG. 5B provides SEM images of MC3T3-E1 cells on PCL scaffold, PCL scaffold-PCG20, and PCL scaffold-BG5 after culture for 2 hours, 4 hours, 12 hours, and 4 days. Scale bar =20 μm. White arrows indicate filopodia.

FIG. 6A provides CLSM images of PCL scaffold, PCL scaffold-BG, and PCL scaffold-BG-BMP2-FITC. BMP2-FITC peptide was introduced via soaking PCL scaffold-BG in 300 μg/mL peptide TBS solution for 24 hours. Scale bar=100 μm. FIG. 6B provides a graph of the cumulative BMP2-FITC release profile from PCL scaffold-BG-BMP2-FITC. FIG. 6C provides a graph of the cumulative release profile of BMP2-FITC peptide from PCL scaffold-BG-BMP2-FITC. BMP2-FITC was incorporated to PCL scaffold-BG by immersing the scaffold in 100 μg/mL peptide TBS solution for 12 hours. FIG. 6D provides graphs of the relative mRNA expression of osteogenic marker genes in rBMSCs after culture on PCL scaffold, PCL scaffold-BG, and PCL scaffold-BG-BMP2 in the osteogenic differentiation medium for 7 days and 21 days. *p<0.05; n=3 per group.

### DETAILED DESCRIPTION OF THE INVENTION

Repairing large tissue defects often represents a great challenge in clinics due to issues regarding lack of donors, mismatched sizes, irregular shapes, and immune rejection. Three-dimensional (3D) printed scaffolds are attractive for growing cells and producing tissue constructs because of the intricate control over pore size, porosity, and geometric shape, but the lack of biomimetic surface nanotopography and limited biomolecule presenting capacity render them less efficacious in regulating cell responses.

Herein, a facile method for coating scaffolds, particularly 3D printed scaffolds, with nanofibers, particularly electrospun nanofiber segments, is reported. The surface morphology of modified 3D scaffolds changes dramatically, displaying a biomimetic nanofibrous structure, while the bulk mechanical property, pore size and porosity are not significantly compromised. The short nanofibers-decorated 3D printed scaffolds significantly promote adhesion and proliferation of cells such as pre-osteoblasts and bone marrow mesenchymal stem cells (BMSCs). Further immobilization of bone morphogenetic protein-2 (BMP-2) mimicking peptides to nanofiber segments-decorated scaffolds show enhanced mRNA expressions of osteogenic markers Runx2, Alp, OCN, and BSP in BMSCs, indicating the enhancement of BMSCs osteogenic differentiation. The approach presented herein greatly expands the functions of scaffolds (e.g., 3D printed scaffolds) and enhances the efficacy of scaffolds for tissue engineering.

Recently, three-dimensional (3D) printing has been developed and serves as a promising technology to produce 3D scaffolds for tissue regeneration (Peltola, et al. (2008) Ann. Med., 40:268; Heinrich, et al. (2019) Small 1805510). While still relatively new, 3D printed scaffolds have many advantages over other approaches, including flexibility in design, capability of generating sophisticated geometric shapes and interconnected pores with high precision (Ngo, et al. (2018) Composites Part B 143:172). High-resolution scaffolds have been configured by industrial 3D printers, and the minimum layer thickness ranges from 16 to 178 μm for different 3D printing systems (Chia, et al. (2015) J. Biol. Eng., 9:4). However, such resolutions are not optimized for building functional, biomimetic scaffolds featuring multiple hierarchies of structures. Scaffolds for tissue repair often necessitate 3D intricate architectures in macro-, micro-, and nanoscales. 3D printing fails to produce surface nanotopographies while providing controllable geometric shape (macro-architecture) and pore size, shape, interconnection, and spatial distribution (micro-architecture). Therefore, surface decoration with functional nanomaterials is desirable to improve the performance of 3D printed scaffolds as tissue engineering constructs.

Electrospinning is a versatile technique which can process different types of materials into filaments with diameters ranging from several nanometers to several microns (Do, et al. (2015) Adv. Healthcare Mater., 4:1742). This technique has been used to fabricate membranes consisting of interlaced fibers (Jiang, et al. (2015) Prog. Polym. Sci., 46:1; Kai, et al. (2014) Mater. Sci. Eng. C-Mater., 45:659; Karuppuswamy, et al., (2014) Appl. Surf. Sci., 322:162). These fibrous membranes capable of mimicking natural extracellular matrix (ECM) architectures provide biophysical and biochemical supports for cells (Caralt, et al. (2015) Am. J. Transplant 15:64; Bonnans, et al. (2014) Nat. Rev. Mol. Cell Biol., 15:786; Xu, et al. (2013) Biomaterials 34:130). Furthermore, the nanofibers are easily functionalized with bioactive molecules such as peptides, proteins, DNAs, and RNAs through either encapsulation or surface immobilization (Hu, et al. (2014) Control J. Release 185:12; Lee, et al. (2011) Acta Biomater., 7:3868; Xu, et al. (2008) Eur. J. Pharm. Biopharm., 70:165; Li, et al. (2010) Colloids Surf. B 75:418; Jiang, et al. (2008) Biomacromolecules 9:2097; Duque, et al. (2016) Biomaterials 106:24; Chen, et al. (2018) Adv. Drug Deliver. Rev., 132:188). However, the fibers produced from conventional electrospinning are closely packed, and pore space shrinks as fiber diameter decreases. The small pore size hinders cell infiltration, resulting in restricted tissue ingrowth (Sisson, et al. (2010) J. Biomed. Mater. Res. A 94A:1312). In addition, the mechanical properties of fibrous scaffolds are usually poor because of the large surface area-to-volume ratios and high porosities.

Herein, it is shown that combining 3D printing and electrospinning makes their respective advantages complementary, increasing the ability to develop functional scaffolds for tissue regeneration. A thin layer of carbon nanotube coatings to triacetate-cellulose bimorph fibers can effectively modulate the infrared radiation upon the change of relative humidity (Zhang, et al. (2019) Science 363:619). Herein, a simple and versatile method to decorate scaffolds, particularly 3D printed scaffolds, with nanofibers, particularly electrospun nanofiber segments, for regulating cellular responses was developed. To demonstrate the proof-of-concept, bioactive glass (BG) nanofibers were selected to decorate 3D printed scaffolds, as BGs are resorbable, can form bioactive hydroxycarbonate apatite (HCA), release ions such as Si to promote osteogenesis, stimulate angiogenic growth factor secretion, and suppress inflammation and bacterial growth (Xie, et al. (2008) Macromol. Rapid Commun., 29:1775; Lepparanta, et al. (2008) J. Mater. Sci. Mater. Med., 19:547; Eberhard, et al. (2005) Biomaterials 26:1545; Bunting, et al. (2005) J. Hand. Surg., 30B:242; Xynos, et al. (2001) J. Biomed. Mater. Res., 55:151; Jell, et al. (2006) J. Mater. Sci. Mater. Med., 17:997; Day, R. M. (2005) Tissue Eng., 11:768). Immobilized E7 domain modified bone morphogenetic protein-2 (BMP2) mimicking peptides were used to the decorated BG nanofibers to enhance regulation of cellular responses through calcium coupling. To show the versatility, poly(lactic-co-glycolic acid) (PLGA)-collagen-gelatin (PCG) nanofiber segments were added to 3D printed scaffolds as they were used to fabricate nanofiber aerogels for cranial bone regeneration and showed an appropriate degradation property without eliciting evident inflammatory response (Weng, et al. (2018) Adv. Healthcare Mater., 7:1701415).

In accordance with the instant invention, surface modified scaffolds (also referred to herein as coated scaffolds) and methods of synthesizing the surface modified scaffolds are provided. In certain embodiments, the surface modified scaffolds allow for cell growth and/or infiltration and/or differentiation. The surface modified scaffolds of the present invention can be designed to have controlled pore size, a biomimetic surface nanotopography, and/or cell adhesion (e.g., temperature-controlled cell adhesion). The surface modified scaffolds of the present invention can be used for a number of purposes including, but not limited to, cell expansion (e.g., in vitro, such as in bioreactors), 3D cell culture, tissue modeling, and tissue regeneration. The surface modified scaffolds of the instant invention may also be used as grafts.

In certain embodiments, the method of synthesizing the surface modified scaffolds of the instant invention comprises coating a scaffold with a coating material (e.g., gelatin) and nanofibers. In certain embodiments, the method comprises treating the scaffold with plasma (e.g., air plasma or oxygen plasma) prior to coating. In certain embodiments, the method comprises coating the scaffold with a solution or suspension comprising gelatin and the nanofibers. In certain embodiments, the method comprises first coating the scaffold with gelatin and then coating with the nanofibers. In certain embodiments, the coated scaffolds are then crosslinked (e.g., chemically crosslinked such as with glutaraldehyde). The methods may further comprise adding further agents or compounds to the coating of the scaffold, either before or after the crosslinking step. The methods may further comprise synthesizing the scaffold (e.g., by 3D printing) prior to coating the scaffold.

The scaffolds of the instant invention may be manufactured or synthesized by any method. For example, the scaffolds of the present invention can be manufactured using a variety of methods including, but not limited to: 3D printing, melt electrospinning, and melt electrospinning writing. In certain embodiments, the scaffold is made by 3D printing such as extrusion-based 3D printing. In certain embodiment, the scaffold is made by melt electrospinning writing (e.g., as a type of 3D printing). Notably, 3D printing allows for controlling the shape of the scaffold, controlling the pore sizes within the scaffold, controlling spatial distribution (e.g., micro-architecture), and/or controlling the interconnections of the scaffold.

The scaffolds of the present invention may comprise any material. For example, the scaffolds may comprise plastics, polymers, ceramic, glass, and/or metal or mixtures thereof. In certain embodiments, the scaffolds comprise at least one polymer.

The scaffold of the instant invention may comprise fibers. For example, the scaffold may comprise microfibers and/or nanofibers. As used herein, nanofibers are fibers having a diameter less than about 1 μm (e.g., average diameter), but greater than about 1 nm. As used herein, microfibers are fibers having a diameter greater than about 1 μm (e.g., average diameter), but less than about 1 millimeter. In certain embodiments, the scaffolds of the instant invention comprise microfibers. In certain embodiments, the microfibers of the scaffold have a diameter (e.g., average diameter) of about 1 μm to about 750, about 1 μm to about 500 μm, about 1 μm to about 100 μm, about 1 μm to about 50 μm, about 10 μm to about 50 μm, about 100 μm to about 500 μm, about 200 μm to about 400 μm, about 250 μm to about 350 μm, or about 300 μm. In certain embodiments, the microfibers of the scaffold have a diameter (e.g., average diameter) of at least about 1 μm, at least about 10 μm, at least about 20 μm, at least about 50 μm, at least about 100 μm, at least about 150 μm, at least about 200 μm, at least about 250 μm, or at least about 300 μm. In certain embodiments, the microfibers of the scaffold have a diameter (e.g., average diameter) of less than about 700 μm, less than about 600 μm, less than about 500 μm, less than about 450 μm, less than about 400 μm, less than about 350 μm, less than about 300 μm, less than about 250 μm, less than about 200 μm, less than about 100 μm, or less than about 50 μm.

In certain embodiments, the scaffolds of the instant invention comprise wells, pores, and/or holes. In certain embodiments, the wells, pores, and/or holes are interconnected. The wells, pores and/or holes may or may not proceed the entire width and/or length through the scaffold. The wells, pores and/or holes may be in alignment or arranged throughout the scaffold or be present randomly throughout the scaffold. In certain embodiments, the wells, pores and/or holes of the scaffold have a diameter (e.g., average diameter) of about 1 μm to about 750, about 50 μm to about 500 μm, about 100 μm to about 500 μm, about 200 μm to about 400 μm, about 250 μm to about 350 μm, or about 300 μm. In certain embodiments, the wells, pores, and/or holes of the scaffold have a diameter (e.g., average diameter) of at least about 1 μm, at least about 50 μm, at least about 100 μm, at least about 150 μm, at least about 200 μm, at least about 250 μm, or at least about 300 μm. In certain embodiments, the wells, pores, and/or holes of the scaffold have a diameter (e.g., average diameter) of less than about 700 μm, less than about 600 μm, less than about 500 μm, less than about 450 μm, less than about 400 μm, less than about 350 μm, or less than about 300 μm. In certain embodiments, the wells, pores, and/or holes cover at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% of the surface of the scaffold. In certain embodiments, the wells, pores, and/or holes cover less than about 95%, less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 10%, less than about 5%, less than about 1%, of the surface of the scaffold.

The fibers of the scaffold may be of any orientation. For example, the scaffold may comprise orthogonal fibers, aligned fibers (e.g., uniaxially aligned), partially aligned, random fibers, and/or entangled fibers. In certain embodiments, the scaffold comprises random fibers. In certain embodiments, the scaffold comprises aligned fibers (e.g., uniaxially, radially, vertically, or horizontally). In certain embodiments, the scaffold comprises aligned fibers. In certain embodiments, the scaffold comprises orthogonal fibers.

As explained hereinabove, the scaffold may comprise a polymer(s), e.g., polymer fibers. The fibers of the scaffolds of the instant invention may comprise any polymer. In certain embodiments, the fibers are electrospun fibers. In a particular embodiment, the polymer is biocompatible. The polymer may be biodegradable or non-biodegradable. In a particular embodiment, the polymer is a biodegradable polymer. The polymer may by hydrophobic, hydrophilic, or amphiphilic. In a particular embodiment, the polymer is hydrophobic. The polymer may be, for example, a homopolymer, random copolymer, blended polymer, copolymer, or a block copolymer. Block copolymers are most simply defined as conjugates of at least two different polymer segments or blocks. The polymer may be, for example, linear, star-like, graft, branched, dendrimer based, or hyper-branched (e.g., at least two points of branching). The polymer of the invention may have from about 2 to about 10,000, about 2 to about 1000, about 2 to about 500, about 2 to about 250, or about 2 to about 100 repeating units or monomers. The polymers of the instant invention may comprise capping termini.

Examples of hydrophobic polymers include, without limitation: poly(hydroxyethyl methacrylate), poly(N-isopropyl acrylamide), poly(lactic acid) (PLA (or PDLA)), poly(lactide-co-glycolide) (PLG), poly(lactic-co-glycolic acid) (PLGA), polyglycolide or polyglycolic acid (PGA), polycaprolactone (PCL), poly(aspartic acid), polyoxazolines (e.g., butyl, propyl, pentyl, nonyl, or phenyl poly(-oxazolines)), polyoxypropylene, poly(glutamic acid), poly(propylene fumarate) (PPF), poly(trimethylene carbonate), polycyanoacrylate, polyurethane, polyorthoesters (POE), polyanhydride, polyester, poly(propylene oxide), poly(caprolactonefumarate), poly(1,2-butylene oxide), poly(n-butylene oxide), poly(ethyleneimine), poly(tetrahydrofurane), ethyl cellulose, polydipyrolle/dicabazole, starch, polyvinylidene fluoride (PVDF), polytetrafluoroethylene (PTFE), polydioxanone (PDO), polyether poly(urethane urea) (PEUU), cellulose acetate, polypropylene (PP), polyethylene terephthalate (PET), nylon (e.g., nylon 6), polycaprolactam, PLA/PCL, poly(3-hydroxybutyrate-co-3-hydroxyvalerate) (PHBV), PCL/calcium carbonate, and/or poly(styrene). In a particular embodiment, the hydrophobic polymer comprises polycaprolactone (PCL).

Examples of hydrophilic polymers include, without limitation: polyvinyl alcohol (PVA), polyvinylpyrrolidone (PVP), poly(ethylene glycol) and poly(ethylene oxide) (PEO), chitosan, collagen, chondroitin sulfate, sodium alginate, gelatin, elastin, hyaluronic acid, silk fibroin, sodium alginate/PEO, silk/PEO, silk fibroin/chitosan, hyaluronic acid/gelatin, collagen/chitosan, chondroitin sulfate/collagen, and chitosan/PEO.

Amphiphilic copolymers or polymer composites may comprise a hydrophilic polymer (e.g., segment) and a hydrophobic polymer (e.g., segment)—such as those listed above (e.g., gelatin/ polyvinyl alcohol (PVA), PCL/collagen, chitosan/PVA, gelatin/elastin/PLGA, PDO/elastin, PHBV/collagen, PLA/hyaluronic acid, PLGA/hyaluronic acid, PCL/hyaluronic acid, PCL/collagen/hyaluronic acid, gelatin/siloxane, PLLA/MWNTs/hyaluronic acid).

In certain embodiments, the fiber comprises polymethacrylate, poly vinyl phenol, polyvinylchloride, cellulose, polyvinyl alcohol, polyacrylamide, poly(lactic-co-glycolic acid) (PLGA), collagen, polycaprolactone, polyurethanes, polyvinyl fluoride, polyamide, silk, nylon, polybenzimidazole, polycarbonate, polyacrylonitrile, polylactic acid, polyethylene-co-vinyl acetate, polyethylene oxide, polyaniline, polystyrene, polyvinylcarbazole, polyethylene terephthalate, polyacrylic acid-polypyrene methanol, poly(2-hydroxyethyl methacrylate), polyether imide, polyethylene gricol, polyethylene glycol, poly(ethylene-co-vinyl alcohol), polyacrylnitrile, polyvinyl pyrrolidone, polymetha-phenylene isophthalamide, gelatin, alginate, chitosan, starch, pectin, cellulose, methylcellulose, sodium polyacrylate, starch-acrylonitrile co-polymers, bioactive glass, and/or combinations of two or more polymers. Multiple polymers may be mixed to form the fibers. The polymers may be mixed in equal ratios or various ratios depending on the desired properties of the nanofibers.

Prior to coating, the scaffold may also be modified (e.g., physically and/or chemically) to enhance the coating process. In certain embodiments, the modification increases the hydrophilicity of the scaffold. In certain embodiments, the scaffold undergoes plasma treatment (e.g., air plasma and/or oxygen plasma). Plasma treatment will generate negatively charged groups (e.g., carboxyl groups) that enhance the interaction between the scaffold and the coating.

The scaffolds of the instant invention may be coated with a coating material such as gelatin. While gelatin is described herein as the coating material, other coating materials may be used (e.g., coating materials with adhesive properties). For example, the scaffolds may be coated with a hydrogel, collagen, a proteoglycans, elastin, a glycosaminoglycan (e.g., hyaluronic acid, heparin, chondroitin sulfate, or keratan sulfate), gelatin, alginate, chitosan, chitin, starch, pectin, cellulose, methylcellulose, sodium polyacrylate, starch-acrylonitrile co-polymers, a glue (bioadhesive) (e.g., fibrin glue), and/or other natural or synthetic hydrogels, and derivatives thereof (e.g., del Valle et al., Gels (2017) 3:27). As used herein, a hydrogel is a polymer matrix able to retain water, particularly large amounts of water, in a swollen state. In certain embodiments, the coating material is gelatin, chitosan, collagen, cellulose, chitin, a hydrogel, or a glue (bioadhesive) (e.g., fibrin glue). In certain embodiments, the coating material is gelatin.

The scaffolds of the instant invention may be coated with multiple layers. When the scaffold is coated with more than one layer, the layers may be different (e.g., comprise different coating materials, agents, nanofibers, and/or different amounts of coating materials) or the same. For examples, the scaffold may be coated with a first layer comprising the coating material and the nanofiber and then coated with a second layer comprising the coating material. When multiple layers are present, the layers may be crosslinked after each coating or crosslinked after more than one (or all) coatings are added.

The term “coat” refers to a layer of a substance/material on the surface of a scaffold and/or the fibers of the scaffold. Coatings may, but need not, also impregnate the scaffold (e.g., form a layer within pores and/or holes). Further, while a coating may cover 100% of the scaffold, a coating may also cover less than 100% of the surface of the scaffold (e.g., at least about 75%, at least about 80%, at least about 85%, at least about 90%, at least about 95%, at least about 98%, or more of the surface may be coated).

The coating material may be applied to the scaffold by any method. For example, the coating material may be applied to the scaffold by immersing or soaking the scaffold in a solution or suspension comprising the coating material, spraying (e.g., electrospraying) the scaffold with a solution or suspension comprising the coating material, and/or physically applying (e.g., painting) a solution or suspension comprising the coating material onto the scaffold. In certain embodiments, the coating material is applied to the scaffold by immersing or soaking the scaffold in a solution or suspension comprising the coating material.

In certain embodiments, the coating material (e.g., gelatin) is contained in a solution at about 0.1 mg/ml to about 10 mg/ml, about 0.5 mg/ml to about 5 mg/ml, about mg/ml to about 4 mg/ml, about 0.5 mg/ml to about 3 mg/ml, about 1 mg/ml to about 2 mg/ml, or about 1.5 mg/ml. In certain embodiments, the solution comprises at least about 0.1 mg/ml, about 0.25 mg/ml, about 0.5 mg/ml, about 0.75 mg/ml, or about 1 mg/ml. In certain embodiments, the solution comprises less than about 5 mg/ml, about 4 mg/ml, about 3 mg/ml, about 2.5 mg/ml, or about 2 mg/ml. In certain embodiments, the solution has an acidic pH. In certain embodiments, the solution comprises acetic acid (e.g., acetic buffer). In certain embodiments, the pH of the solution is about 3 to about 6, about 3 to about 5, about 3.5 to about 4.5, about 3.75 to about 4.25, or about 4. In certain embodiments, the pH is at least about 2, at least about 3, at least about 3.25 at least about 3.5, at least about 3.75, or at least about 4. In certain embodiments, the pH is less than about 6, less than about 5, less than about 4.75, less than about 4.5, less than about 4.25, or less than about 4.

In certain embodiments, the coating material can be formed by polymerization (e.g., with a thermosensitive hydrogel). For example, the coating material may comprise poly(N-isopropylacrylamide) (PNIPAAm), which is a temperature responsive polymer. Upon increasing the temperature (e.g., to about 32° C.±5-10° C.) of a solution (e.g., aqueous solution) comprising PNIPAAm, polymerization occurs and the scaffold (e.g., immersed or soaked in the solution) becomes coated with the coating material.

The coating material and optionally the nanofibers may be crosslinked (e.g., with the scaffold). Crosslinking may be done using a variety of techniques including thermal crosslinking, chemical crosslinking, UV-crosslinking, and photo-crosslinking. For example, the coated scaffold of the instant invention may be crosslinked with a crosslinker such as, without limitation: formaldehyde, paraformaldehyde, acetaldehyde, glutaraldehyde, a photocrosslinker, genipin, and natural phenolic compounds (Mazaki, et al., Sci. Rep. (2014) 4:4457; Bigi, et al., Biomaterials (2002) 23:4827-4832; Zhang, et al., Biomacromolecules (2010) 11:1125-1132; incorporated herein by reference). The crosslinker may be a bifunctional, trifunctional, or multifunctional crosslinking reagent. In certain embodiments, the crosslinker is glutaraldehyde. In certain embodiments, the crosslinking is performed using UV crosslinking. When multiple coats or layers are used, the individual coats or layers may be crosslinked before the addition of the next layer or the other layers may be added prior to crosslinking after the addition of additional layers.

The nanofibers of the present invention may comprise any material. For example, the nanofibers may comprise plastics, polymers, ceramic, glass, and/or metal or mixtures thereof. In certain embodiments, the nanofibers comprise at least one polymer. In certain embodiments, the nanofibers are electrospun nanofibers. In certain embodiments, the nanofibers comprise bioactive glass. While the nanofibers coating the surface of the scaffolds of the instant invention are described as nanofibers, the scaffolds of the instant invention may be coated with microfibers either instead of nanofibers or in addition to the nanofibers.

The nanofibers of the instant invention may comprise any polymer. In a particular embodiment, the polymer is biocompatible. The polymer may be biodegradable or non-biodegradable. In a particular embodiment, the polymer is a biodegradable polymer. The polymer may by hydrophobic, hydrophilic, or amphiphilic. In a particular embodiment, the polymer is hydrophobic. The polymer may be, for example, a homopolymer, random copolymer, blended polymer, copolymer, or a block copolymer. Block copolymers are most simply defined as conjugates of at least two different polymer segments or blocks. The polymer may be, for example, linear, star-like, graft, branched, dendrimer based, or hyper-branched (e.g., at least two points of branching). The polymer of the invention may have from about 2 to about 10,000, about 2 to about 1000, about 2 to about 500, about 2 to about 250, or about 2 to about 100 repeating units or monomers. The polymers of the instant invention may comprise capping termini.

Examples of hydrophobic polymers include, without limitation: poly(hydroxyethyl methacrylate), poly(N-isopropyl acrylamide), poly(lactic acid) (PLA (or PDLA)), poly(lactide-co-glycolide) (PLG), poly(lactic-co-glycolic acid) (PLGA), polyglycolide or polyglycolic acid (PGA), polycaprolactone (PCL), poly(aspartic acid), polyoxazolines (e.g., butyl, propyl, pentyl, nonyl, or phenyl poly(-oxazolines)), polyoxypropylene, poly(glutamic acid), poly(propylene fumarate) (PPF), poly(trimethylene carbonate), polycyanoacrylate, polyurethane, polyorthoesters (POE), polyanhydride, polyester, poly(propylene oxide), poly(caprolactonefumarate), poly(1,2-butylene oxide), poly(n-butylene oxide), poly(ethyleneimine), poly(tetrahydrofurane), ethyl cellulose, polydipyrolle/dicabazole, starch, polyvinylidene fluoride (PVDF), polytetrafluoroethylene (PTFE), polydioxanone (PDO), polyether poly(urethane urea) (PEUU), cellulose acetate, polypropylene (PP), polyethylene terephthalate (PET), nylon (e.g., nylon 6), polycaprolactam, PLA/PCL, poly(3-hydroxybutyrate-co-3-hydroxyvalerate) (PHBV), PCL/calcium carbonate, and/or poly(styrene). In a particular embodiment, the hydrophobic polymer comprises polycaprolactone (PCL).

Examples of hydrophilic polymers include, without limitation: polyvinyl alcohol (PVA), polyvinylpyrrolidone (PVP), poly(ethylene glycol) and poly(ethylene oxide) (PEO), chitosan, collagen, chondroitin sulfate, sodium alginate, gelatin, elastin, hyaluronic acid, silk fibroin, sodium alginate/PEO, silk/PEO, silk fibroin/chitosan, hyaluronic acid/gelatin, collagen/chitosan, chondroitin sulfate/collagen, and chitosan/PEO.

Amphiphilic copolymers or polymer composites may comprise a hydrophilic polymer (e.g., segment) and a hydrophobic polymer (e.g., segment)—such as those listed above (e.g., gelatin/polyvinyl alcohol (PVA), PCL/collagen, chitosan/PVA, gelatin/elastin/PLGA, PDO/elastin, PHBV/collagen, PLA/hyaluronic acid, PLGA/hyaluronic acid, PCL/hyaluronic acid, PCL/collagen/hyaluronic acid, gelatin/siloxane, PLLA/MWNTs/hyaluronic acid).

Examples of polymers particularly useful for electrospinning are provided in Xie et al. (Macromol. Rapid Commun. (2008) 29:1775-1792; incorporated by reference herein; see e.g., Table 1). Examples of compounds or polymers for use in nanofibers, particularly for electrospun nanofibers include, without limitation: natural polymers (e.g., chitosan, gelatin, collagen type I, II, and/or III, elastin, hyaluronic acid, cellulose, silk fibroin, phospholipids (Lecithin), fibrinogen, hemoglobin, fibrous calf thymus Na-DNA, virus M13 viruses), synthetic polymers (e.g., PLGA, PLA, PCL, PHBV, PDO, PGA, PLCL, PLLA-DLA, PEUU, cellulose acetate, PEG-b-PLA, EVOH, PVA, PEO, PVP), blended (e.g., PLA/PCL, gelatin/PVA, PCL/gelatin, PCL/collagen, sodium alginate/PEO, chitosan/PEO, Chitosan/PVA, gelatin/elastin/PLGA, silk/PEO, silk fibroin/chitosan, PDO/elastin, PHBV/collagen, hyaluronic acid/gelatin, collagen/chondroitin sulfate, collagen/chitosan), and composites (e.g., PDLA/HA, PCL/CaCO₃, PCL/HA, PLLA/HA, gelatin/HA, PCL/collagen/HA, collagen/HA, gelatin/siloxane, PLLA/MWNTs/HA, PLGA/HA).

In certain embodiments, the nanofibers comprise polymethacrylate, poly vinyl phenol, polyvinylchloride, cellulose, polyvinyl alcohol, polyacrylamide, poly(lactic-co-glycolic acid) (PLGA), collagen, polycaprolactone, polyurethanes, polyvinyl fluoride, polyamide, silk, nylon, polybenzimidazole, polycarbonate, polyacrylonitrile, polylactic acid, polyethylene-co-vinyl acetate, polyethylene oxide, polyaniline, polystyrene, polyvinylcarbazole, polyethylene terephthalate, polyacrylic acid-polypyrene methanol, poly(2-hydroxyethyl methacrylate), polyether imide, polyethylene gricol, polyethylene glycol, poly(ethylene-co-vinyl alcohol), polyacrylnitrile, polyvinyl pyrrolidone, polymetha-phenylene isophthalamide, gelatin, alginate, chitosan, starch, pectin, cellulose, methylcellulose, sodium polyacrylate, starch-acrylonitrile co-polymers, bioactive glass, and/or combinations of two or more polymers. Multiple polymers may be mixed to form the fibers. The polymers may be mixed in equal ratios or various ratios depending on the desired properties of the nanofibers.

In certain embodiments, the nanofiber comprises poly(lactic-co-glycolic acid) (PLGA). In certain embodiments, the nanofiber comprises poly(lactic-co-glycolic acid) (PLGA)-collagen. In certain embodiments, the nanofiber comprises poly(lactic-co-glycolic acid) (PLGA)-collagen-gelatin. In certain embodiments, the nanofiber comprises a polymer (e.g., a hydrophobic polymer) and the coating material (e.g., gelatin).

In certain embodiments, the nanofibers are mineralized (e.g., comprise minerals and/or coated with minerals). Mineralization, for example, with hydroxyapatite, can enhance the adhesion of osteogenic precursor cells in vitro and in vivo (Duan, et al., Biomacromolecules (2017) 18:2080-2089). In certain embodiments, the nanofibers are coated with Ca, P, and/or O. In certain embodiments, the nanofibers are coated with hydroxyapatite, fluorapatite, and/or chlorapatite, particularly hydroxyapatite. In certain embodiments, the nanofibers are immersed in simulated body fluid (SBF) for the mineralization (e.g., a solution comprising NaC₁, CaCl₂, NaH₂PO₄, and NaHCO₃). The nanofibers may be mineralized before and/or after coating onto the scaffold.

In certain embodiments, the nanofibers comprise bioactive glass (e.g., bioactive glass nanofibers). Bioactive glasses are biologically compatible synthetic materials comprising varying amounts of silicates/silica (e.g., SiO₂), sodium oxides (e.g., NaO₂), calcium oxides (e.g., CaO), and/or phosphates/phosphorus pentoxide (e.g., P₂O₅). In certain embodiments, the bioactive glass comprises silicates/silica (e.g., SiO₂), calcium oxides (e.g., CaO), and phosphates/phosphorus pentoxide (e.g., P₂O₅). In certain embodiments, the molar ratio of Si:P:Ca is 70-90:5-15:5-15, 75-85:7.5-12.5:7.5-12.5, or about 80:10:10.

In certain embodiments, the nanofibers of the instant invention are nanofiber segments. The nanofiber segments can be synthesized by any method, but are typically derived from longer nanofibers (e.g., electrospun nanofibers). For example, nanofiber segments may be derived from nanofibers by cutting, breaking, cryocutting (e.g., with a cryotome), wet milling, cryomilling, and/or homogenization (e.g., by sonication, particularly probe sonication). In certain embodiments, the nanofibers are cryo-cut. For example, the nanofibers may be frozen (e.g., in a liquid such as water (e.g., at about −20° C. or lower or at about −80° C. or lower)) and then the frozen block containing the nanofibers is cut with a cryotome (e.g., at about −20° C. or lower). In certain embodiments, the nanofibers can be broken or homogenized into nanofiber segments by sonication. For example, the nanofibers can be placed into water or an aqueous solution and homogenized using an ultrasonic probe sonicator (e.g., equipped with a microtip probe (e.g., ⅛ mm)). In certain embodiments, the nanofibers are cut into nanofiber segments using cryomilling (e.g., in liquid nitrogen).

In certain embodiments, the nanofiber segments are less than about 150 μm in length, less than about 100 μm in length, less than about 75 μm in length, less than about μm in length, less than about 40 μm in length, less than about 30 μm in length, less than about 25 μm in length, less than about 20 μm in length, less than about 15 μm in length, or less than about 10 μm in length. In certain embodiments, the nanofiber segments have a median or mean length of about 0.1 μm to about 100 μm in length, about 0.1 μm to about 50 μm in length, about 5 μm to about 50 μm in length, about 0.5 μm to about 25 μm in length, about 1 μm to about 20 μm in length, about 1 μm to about μm in length, about 1 μm to about 10 μm in length, or about 5 μm to about 15 μm in length.

The nanofibers may be applied to the scaffold by any method. For example, the nanofibers may be applied to the scaffold by immersing or soaking the scaffold in a solution or suspension comprising the nanofibers, spraying (e.g., electrospraying) the scaffold with a solution or suspension comprising the nanofibers, and/or physically applying (e.g., painting) a solution or suspension comprising the nanofibers onto the scaffold. In certain embodiments, the nanofibers is applied to the scaffold by immersing or soaking the scaffold in a solution or suspension comprising the nanofibers, optionally further comprising the coating material. In certain embodiments, the solution or suspension comprising the nanofibers further comprises the coating material and/or agent. In certain embodiments, the nanofibers cover less than about 95%, less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 10%, less than about 5%, less than about 1%, of the surface area of the scaffold. In certain embodiments, the nanofibers cover about 30% to about 70% or about 40% to about 60% of the surface area of the scaffold.

In certain embodiments, the nanofiber is contained in a solution or solution at about 0.1 mg/ml to about 100 mg/ml, about 0.5 mg/ml to about 50 mg/ml, about 1 mg/ml to about 40 mg/ml, about 1 mg/ml to about 30 mg/ml, or about 5 mg/ml to about 20 mg/ml. In certain embodiments, the solution or suspension has an acidic pH. In certain embodiments, the solution or suspension comprises acetic acid (e.g., acetic buffer). In certain embodiments, the pH of the solution or suspension is about 3 to about 6, about 3 to about 5, about 3.5 to about 4.5, about 3.75 to about 4.25, or about 4. In certain embodiments, the pH is at least about 2, at least about 3, at least about 3.25 at least about 3.5, at least about 3.75, or at least about 4. In certain embodiments, the pH is less than about 6, less than about 5, less than about 4.75, less than about 4.5, less than about 4.25, or less than about 4.

After synthesis, the coated scaffolds may be washed or rinsed in water and/or a desired carrier or buffer (e.g., a pharmaceutically or biologically acceptable carrier). The coated scaffolds may also be stored in a cold solution, lyophilized and/or freeze-dried. The coated scaffolds may also be physically manipulated such as by compressing and/or shaping or trimming of the coated scaffolds (e.g., to achieve a desired shape).

The coated scaffolds of the instant invention may also be sterilized. For example, the coated scaffolds can be sterilized using various methods (e.g., by treating with ethylene oxide gas, gamma irradiation, or 70% ethanol).

The coated scaffolds of the instant invention may comprise and/or encapsulate cells or tissue (e.g., the coated scaffolds may be seeded with cells (e.g., in the wells, holes, and/or pores). In a particular embodiment, the cells are autologous to the subject to be treated with the coated scaffold. The coated scaffolds may comprise and/or encapsulate any cell type. Cell types include, without limitation: embryonic stem cells, adult stem cells, bone marrow stem cells, induced pluripotent stem cells, progenitor cells (e.g., neural progenitor cells), embryonic like stem cells, mesenchymal stem cells, bone marrow mesenchymal stem cells, CAR-T cells, immune cells (including but not limited to T cells, B cells, NK cells, macrophages, neutrophils, dendritic cells and modified forms of these cells and various combinations thereof), cell based vaccines, and cell lines expressing desired therapeutic proteins and/or genes. In a particular embodiment, the cells comprise stem cells. In a particular embodiment, the cells comprise bone marrow stem cells (e.g., BMCSs). In a particular embodiment, the cells comprise dermal fibroblasts. In a particular embodiment, the coated scaffold comprises and/or encapsulates cell spheroids. In a particular embodiment, the coated scaffold comprises and/or encapsulates tissue samples (e.g., minced tissue), such as skin tissue samples or bone samples. The cells or tissue may be cultured within the coated scaffolds (e.g., the cells or tissue may be cultured for sufficient time to allow for growth within and/or infiltration into the coated scaffold). For example, the cells or tissue may be cultured in the coated scaffold for 1, 2, 3, 4, 5, 6, 7, 8, 9, 10, 11, 12, 13, 14, 15, 16, 17, 18, 19, 20, or more days. In certain embodiments, the cells or tissue may be cultured within the coated scaffolds in differentiation media. In a particular embodiment, the cells or tissue may be seeded into the coated scaffold under a vacuum. In a particular embodiment, the coated scaffold is in solution with the cells or tissue and a vacuum is applied, thereby resulting in the seeding of the cells or tissue within the coated scaffold.

The coated scaffolds of the instant invention may comprise or encapsulate at least one agent, particularly a bioactive agent, biologic, cell based therapy, tissue based therapy, and/or drug. In certain embodiments, the agent is added to the coating material, before and/or after crosslinking. In certain embodiments, the agent is linked to the nanofiber. In certain embodiments, the agent is hydrophilic. The agent may be added to the coated scaffold during synthesis and/or after synthesis. The agent may be conjugated (e.g., directly or via a linker) to the nanofiber and/or coating material, encapsulated by the coated scaffold, and/or coated on the coated scaffold (e.g., with, underneath, and/or on top of the coating of the coated scaffold). In a particular embodiment, the agent is not directly conjugated to the coated scaffold (e.g., it is encapsulated within the coating material). In a particular embodiment, the agent is conjugated or linked to the coated scaffold (e.g., surface conjugation or coating). In a particular embodiment, the agents are administered with but not incorporated into the coated scaffold.

The agent may be applied to the scaffold by any method. For example, the agent may be applied to the scaffold by immersing or soaking the scaffold in a solution or suspension comprising the agent, spraying (e.g., electrospraying) the scaffold with a solution or suspension comprising the agent, and/or physically applying (e.g., painting) a solution or suspension comprising the agent onto the scaffold. In certain embodiments, the agent is applied to the scaffold by immersing or soaking the scaffold in a solution or suspension comprising the agent, optionally further comprising the coating material and/or nanofiber. In certain embodiments, the solution or suspension comprising the agent further comprises the coating material and/or nanofiber.

Biologics include but are not limited to small molecules, proteins, peptides, antibodies, antibody fragments, nucleic acid, DNA, RNA, and other known biologic substances, particularly those that have therapeutic use. In a particular embodiment, the agent is a drug or therapeutic agent (e.g., a small molecule) (e.g., analgesic, growth factor, anti-inflammatory, signaling molecule, growth factor, cytokine, antimicrobial (e.g., antibacterial, antibiotic, antiviral, and/or antifungal), hormone (e.g., insulin), ephrins, hemostatic agent (e.g., blood clotting agent, factor, or protein), pain medications (e.g., anesthetics), etc.). In a particular embodiment, the agent enhances tissue regeneration, tissue growth, and wound healing (e.g., growth factors). In a particular embodiment, the agent treats/prevents infections (e.g., antimicrobials such as antibacterials, antivirals and/or antifungals). In a particular embodiment, the agent is an antimicrobial, particularly an antibacterial. In a particular embodiment, the agent enhances wound healing and/or enhances tissue regeneration (e.g., bone, tendon, cartilage, skin, nerve, and/or blood vessel). Such agents include, for example, growth factors, cytokines, chemokines, immunomodulating compounds, and small molecules. Growth factors include, without limitation: platelet derived growth factors (PDGF), vascular endothelial growth factors (VEGF), epidermal growth factors (EGF), neuregulins, fibroblast growth factors (FGF; e.g., basic fibroblast growth factor (bFGF)), insulin-like growth factors (IGF-1 and/or IGF-2), bone morphogenetic proteins (e.g., BMP-2, BMP-7, BMP-12, BMP-9; particularly BMP-2 fragments, peptides, and/or analogs thereof), transforming growth factors (e.g., TGFα, TGFβ, TGFβ3), tumour Necrosis Factor alpha (TNF alpha), nerve growth factors (NGF), neurotrophic factors, stromal derived factor-1 (SDF-1), granulocyte-macrophage colony-stimulating factor (GM-CSF), macrophage colony-stimulating factor (M-CSF), granulocyte-colony stimulating factor (G-CSF), erythropoietin (EPO), glial cell-derived neurotrophic factors (GDNF), hepatocyte growth factors (HGF), keratinocyte growth factors (KGF), neurotrophin, and/or growth factor mimicking peptides (e.g., VEGF mimicking peptides). In a particular embodiment, the growth factor is bFGF. Chemokines include, without limitation: CCL21, CCL22, CCL2, CCL3, CCL5, CCL7, CCL8, CCL13, CCL17, CXCL9, CXCL10, and CXCL11. Cytokines include without limitation IL-2 subfamily cytokines, interferon subfamily cytokines, IL-10 subfamily cytokines, IL-1, I-18, IL-17, tumor necrosis factor, and transforming-growth factor beta superfamily cytokines. Examples of small molecule drugs/therapeutic agents include, without limitation, simvastatin, kartogenin, retinoic acid, paclitaxel, vitamins (e.g., vitamin D3), etc. In a particular embodiment, the agent is a blood clotting factor such as thrombin or fibrinogen. In a particular embodiment, the agent is a bone morphogenetic protein (e.g., BMP-2, BMP-7, BMP-12, BMP-9; particularly human; particularly BMP-2 fragments, peptides, and/or analogs thereof). In a particular embodiment, the agent is a BMP-2 peptide such as KIPKASSVPTELSAISTLYL (SEQ ID NO: 12). In a particular embodiment, the agent is a BMP-2 fragment (e.g., up to about 25, about 30, about 35, about 40, about 45, about 50 amino acids, or more of BMP-2) comprising the knuckle epitope (e.g., amino acids 73-92 of BMP-2 or SEQ ID NO: 12). In a particular embodiment, the BMP-2 peptide is linked to a peptide of acidic amino acids (e.g., Asp and/or Glu; particularly about 3-10 or 5-10 amino acids such as E7, E8, D7, D8) and/or bisphosphonate (e.g., at the N-terminus).

Antimicrobials may include, without limitation, small molecules, peptides, proteins, DNA, RNA, and other known biologic substances. In a particular embodiment, the antimicrobial is a small molecule. In a particular embodiment, the antimicrobial is an antiviral, antifungal, antibiotic or antibacterial, particularly an antibiotic or antibacterial. Examples of antimicrobials include, without limitation, antibiotics such as beta-lactams (e.g., penicillin, ampicillin, oxacillin, cloxacillin, methicillin, cephalosporin, etc.), monobactams (e.g., aztreonam, tigemonam, nocardicin A, tabtoxin, etc.), carbapenems (e.g., imipenem, meropenem, ertapenem, doripenem, etc.), cephalosporins (e.g., cefdinir, cefaclor, cephalexin, cefixime, cefepime, etc.), carbacephems, cephamycins, macrolides (e.g., erythromycin, clarithromycin, azithromycin etc.), quinolones or fluoroquinolones (e.g., ciprofloxacin, levofloxacin, ofloxacin, delafloxacin, etc.), tetracyclines (e.g., tetracycline, doxycycline etc.), sulfonamides (e.g., sulfamethoxazole, sulfafuraxole, etc.), aminoglycosides (e.g., gentamicin, neomycin, tobramycin, kanamycin, etc.), oxazolidinones (e.g., linezolid, posizolid, tedizolid, radezolid, contezolid, etc.), lipopeptides (e.g., daptomycin), glycylcyclines (e.g., tigecycline), moenomycins, aminocoumarins (e.g., novobiocin), co-trimoxazoles (e.g., trimethoprim and sulfamethoxazole), lincosamides (e.g., clindamycin and lincomycin), polypeptides (e.g., colistin), and glycopeptides (e.g., vancomycin); silver containing compounds (e.g., silver ions, silver nitrate, silver nanoparticles, colloidal silver, etc.), gallium containing compounds (e.g., gallium ions, gallium nitrate, gallium nanoparticles, colloidal gallium, etc.), and antimicrobial peptides. Examples of antifungals include, without limitation, amphotericin B, pyrimethamine, thiazoles, allylamines, flucytosine, caspofungin acetate, fluconazole, griseofulvin, terbinafine, amorolfine, imidazoles, triazoles (e.g., voriconazole), flutrimazole, cilofungin, echinocandines, pneumocandin omoconazole terconazole, nystatin, natamycin, griseofulvin, ciclopirox, naftifine, and itraconazole. In a particular embodiment, the antimicrobial is an antibiotic. In a particular embodiment, the antimicrobial is an antimicrobial peptide. In a particular embodiment, the coated scaffold comprises an antimicrobial peptide and at least one other antimicrobial (e.g., antibiotic). Antimicrobial peptides may be therapeutically effective against one or more bacteria. Examples of antimicrobial peptides are provided in the Antimicrobial Peptide Database (aps.unmc.edu/AP/main.php). Examples of antimicrobial peptides are also disclosed in U.S. Pat. Nos. 7,465,784, 9,580,472, 10,144,767, U.S. Patent Application Publication No. 20090156499, U.S. Patent Application Publication No. 20150259382, U.S. Patent Application Publication No. 20140303069, and PCT/US2019/039792, each incorporated by reference herein. In a particular embodiment, the antimicrobial peptide has fewer than about 50 amino acids, fewer than about 25 amino acids, fewer than about 20 amino acids, fewer than about 17 amino acids, fewer than about 15 amino acids, fewer than 12 amino acids, fewer than 10 amino acids, or fewer than 9 amino acids. In a particular embodiment, the antimicrobial peptide has more than about 6 amino acids, particularly more than about 7 amino acids.

Coated scaffolds synthesized by the methods of the instant invention are also encompassed herein. Compositions comprising the coated scaffolds of the instant invention and a carrier (e.g., a pharmaceutically acceptable carrier) are also encompassed herein.

The coated scaffolds of the instant invention can be used to create tissue architectures for a variety of application including, without limitation: wound healing, tissue engineering, tissue growth, tissue repair, tissue regeneration, and engineering 3D in vitro tissue models. Some examples of uses for the coated scaffolds of the instant invention include, but are not limited to: use as tissue structures (in vitro or in vivo), hemostatic bandages, tissue repair structures, and tissue regeneration structures.

The coated scaffolds can also be combined with a variety of hydrogels or biological matrices/cues to form 3D hybrid structures that can release biologically functional agents. The tissue constructs can be used for regeneration of many tissue defects (e.g., skin, bone, cartilage) and healing of various wounds (e.g., injuries, diabetic wounds, venous ulcer, pressure ulcer, burns). The coated scaffolds may be used ex vivo to generate tissue or tissue constructs/models. The coated scaffolds may also be used in vivo in patients (e.g., human or animal) for the treatment of various diseases, disorders, and wounds. In a particular embodiment, the coated scaffold stimulates the growth of existing tissue and/or repair of a wound or defect when applied in vivo. The coated scaffolds can be used for engineering, growing, and/or regeneration of a variety of tissues including but not limited to skin, bone, cartilage, muscle, nervous tissue, and organs (or portions thereof).

In accordance with the instant invention, the coated scaffolds may be used in inducing and/or improving/enhancing wound healing and inducing and/or improving/enhancing tissue regeneration. The coated scaffolds of the present invention can be used for the treatment, inhibition, and/or prevention of any injury or wound. In a particular embodiment, the method comprises administering a coated scaffold comprising an agent and/or cell as described herein. Coated scaffolds of the instant invention can be loaded with different cell types as necessary for regeneration of various tissues. In a particular embodiment, the coated scaffold comprises blood clotting factors (e.g., for accelerating blood clot formation and/or preventing blood loss). For example, the coated scaffold can be used to induce, improve, or enhance wound healing associated with surgery (including non-elective (e.g., emergency) surgical procedures or elective surgical procedures). Elective surgical procedures include, without limitation: liver resection, partial nephrectomy, cholecystectomy, vascular suture line reinforcement and neurosurgical procedures. Non-elective surgical procedures include, without limitation: severe epistaxis, splenic injury, liver fracture, cavitary wounds, minor cuts, punctures, gunshot wounds, and shrapnel wounds. The coated scaffold of the present invention can also be incorporated into delivery devices that allow for their injection/delivery directly into a desired location (e.g., a wound). The coated scaffolds also may be delivered directly into a cavity (such as the peritoneal cavity) (e.g., using a pressurized cannula). In accordance with the instant invention, the coated scaffolds of the present invention can be used to treat and/or prevent a variety of diseases and disorders. Examples of diseases and/or disorders include but are not limited to wounds, ulcers, infections, hemorrhage, tissue injury, tissue defects, tissue damage, bone fractures, bone degeneration, cartilage damage, cancer (e.g., the use of docetaxel and curcumin for the treatment of colorectal cancer (Fan, et al., Sci. Rep. (2016) 6:28373)), neurologic diseases (e.g., Alzheimer's and Parkinson's), ischemic diseases, inflammatory diseases and disorders, heart disease, myocardial infarction, and stroke. Methods for inducing and/or improving/enhancing wound healing in a subject are also encompassed by the instant invention. Methods of inducing and/or improving/enhancing tissue regeneration (e.g., blood vessel growth, neural tissue regeneration, and bone and/or cartilage regeneration) in a subject are also encompassed by the instant invention. Methods of inducing and/or improving/enhancing hemostasis in a subject are also encompassed by the instant invention. The methods of the instant invention comprise administering or applying coated scaffolds of the instant invention to the subject (e.g., at or in a wound). In a particular embodiment, the method comprises administering coated scaffolds comprising an agent and/or cell as described herein. Coated scaffolds of the instant invention can be loaded with different cell types as necessary for regeneration of various tissues. In a particular embodiment, the coated scaffolds comprise blood clotting factors (e.g., for accelerating blood clot formation and/or preventing blood loss). In a particular embodiment, the method comprises administering coated scaffolds to the subject and an agent as described herein (i.e., the agent is not contained within the coated scaffold). When administered separately, the coated scaffold may be administered simultaneously and/or sequentially with the agent. The methods may comprise the administration of one or more coated scaffold. When more than one coated scaffold is administered, the coated scaffolds may be administered simultaneously and/or sequentially.

The coated scaffolds can also be used to expand and increase cell numbers (e.g., stem cell numbers) in culture. In a particular embodiment, microtissues can be grown in situ by prolonged culture of cell laden coated scaffolds (e.g., in confined microfluidic channel devices). These microtissues are injectable or transplantable into a tissue defect to promote wound healing in a subject (e.g., the coated scaffolds comprise autologous cells).

The coated scaffolds may also be employed for cell detection, separation, and/or isolation of cell populations in a mixture. For example, structures conjugated to specific antibodies can be used for the isolation, separation, and/or expansion of different cell types from their mixtures (Custodio, et al., Biomaterials (2015) 43:23-31). Further, coated scaffolds can be used for the in vitro adhesion, proliferation, and/or maturation of chondrocytes as well as in vivo cartilage formation and osteochondral repair induced by coated scaffolds when together with chondrocytes (Liu, et al., Nat. Mater. (2011) 10:398-406).

The coated scaffolds of the present invention may be administered by any method. The coated scaffolds described herein may be administered to a subject or a patient as a pharmaceutical composition. The compositions of the instant invention comprise a coated scaffold and a pharmaceutically acceptable carrier. The term “patient” as used herein refers to human or animal subjects. These compositions may be employed therapeutically, under the guidance of a physician.

The compositions of the instant invention may be conveniently formulated for administration with any pharmaceutically acceptable carrier(s). For example, the agents may be formulated with an acceptable medium such as water, buffered saline, ethanol, polyol (for example, glycerol, propylene glycol, liquid polyethylene glycol and the like), dimethyl sulfoxide (DMSO), oils, detergents, suspending agents or suitable mixtures thereof. Except insofar as any conventional media or agent is incompatible with the agents to be administered, its use in the pharmaceutical preparation is contemplated.

Compositions of the instant invention may be administered by any method. For example, the compositions of the instant invention can be administered, without limitation, parenterally, subcutaneously, orally, topically (ex. using a cream or spray), pulmonarily, rectally, vaginally, intravenously, intraperitoneally, intrathecally, intracerbrally, epidurally, intramuscularly, intradermally, intratumoral, intracarotidly, or by direct injection (e.g., a localized injection into a specific tissue or organ). Selection of a suitable pharmaceutical preparation will also depend upon the mode of administration chosen. For example, the compositions of the invention may be administered parenterally. In this instance, a pharmaceutical preparation comprises the coated scaffolds dispersed in a medium that is compatible with the parenteral injection.

Pharmaceutical compositions containing an agent of the present invention as the active ingredient in intimate admixture with a pharmaceutically acceptable carrier can be prepared according to conventional pharmaceutical compounding techniques.

In a particular embodiment of the instant invention, methods for modulating (increasing) hemostasis; inhibiting blood or cartilage loss; and/or treating hemorrhage are provided. In a particular embodiment, the method comprises administering the coated scaffold to the wound or site of bleeding. In a particular embodiment, the coated scaffolds comprise a blood clotting factor such as thrombin and/or fibrinogen.

In a particular embodiment of the instant invention, methods for stimulating bone and/or cartilage regeneration and/or treating bone and/or cartilage loss are provided. In a particular embodiment, the method comprises administering the coated scaffolds to the site of bone and/or cartilage loss. In a particular embodiment, the site of bone and/or cartilage loss is periodontal. In a particular embodiment, the coated scaffolds are mineralized. In a particular embodiment, the coated scaffolds comprise a bone growth stimulating growth factor such as a bone morphogenic protein or fragment or analog thereof In a particular embodiment, the agent is a bone morphogenetic protein (e.g., BMP-2, BMP-7, BMP-12, BMP-9; particularly human; particularly BMP-2 fragments, peptides, and/or analogs thereof). In a particular embodiment, the agent is a BMP-2 peptide such as KIPKASSVPTELSAISTLYL (SEQ ID NO: 12). In a particular embodiment, the agent is a BMP-2 fragment (e.g., up to about 25, about 30, about 35, about 40, about 45, about 50 amino acids, or more of BMP-2) comprising the knuckle epitope (e.g., amino acids 73-92 of BMP-2 or SEQ ID NO: 12). In a particular embodiment, the BMP-2 peptide is linked to a peptide of acidic amino acids (e.g., Asp and/or Glu; particularly about 3-10 or 5-10 amino acids such as E7, E8, D7, D8) and/or bisphosphonate (e.g., at the N-terminus).

In accordance with the instant invention, antimicrobial (e.g., antibiotic)-loaded coated scaffolds are provided. In a particular embodiment, the antimicrobial (e.g., antibiotic)-loaded coated scaffold is in the form of a wound dressing. The antimicrobial (e.g., antibiotic)-loaded coated scaffolds may be in any form including, without limitation, a wound dressing, bandage, gauze, covering, suture, thread, ligature, hemostasis material, or coating for biomedical device or implant. In a particular embodiment, the antimicrobial (e.g., antibiotic)-loaded coated scaffold is in a wound dressing.

### Definitions

The singular forms “a,” “an,” and “the” include plural referents unless the context clearly dictates otherwise.

As used herein, the term “electrospinning” refers to the production of fibers (i.e., electrospun fibers), particularly micro- or nano-sized fibers, from a solution or melt using interactions between fluid dynamics and charged surfaces (e.g., by streaming a solution or melt through an orifice in response to an electric field). Forms of electrospun nanofibers include, without limitation, branched nanofibers, tubes, ribbons and split nanofibers, nanofiber yarns, surface-coated nanofibers (e.g., with carbon, metals, etc.), nanofibers produced in a vacuum, and the like. The production of electrospun fibers is described, for example, in Gibson et al. (1999) AlChE J., 45:190-195.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., polysorbate 80), emulsifier, buffer (e.g., TrisHCl, acetate, phosphate), water, aqueous solutions, oils, bulking substance (e.g., lactose, mannitol), excipient, auxiliary agent or vehicle with which an active agent of the present invention is administered. Suitable pharmaceutical carriers are described in “Remington's Pharmaceutical Sciences” by E. W. Martin (Mack Publishing Co., Easton, PA); Gennaro, A. R., Remington: The Science and Practice of Pharmacy, (Lippincott, Williams and Wilkins); Liberman, et al., Eds., Pharmaceutical Dosage Forms, Marcel Decker, New York, N.Y.; and Kibbe, et al., Eds., Handbook of Pharmaceutical Excipients (3rd Ed.), American Pharmaceutical Association, Washington.

As used herein, the term “polymer” denotes molecules formed from the chemical union of two or more repeating units or monomers. The term “block copolymer” most simply refers to conjugates of at least two different polymer segments, wherein each polymer segment comprises two or more adjacent units of the same kind.

“Hydrophobic” designates a preference for apolar environments (e.g., a hydrophobic substance or moiety is more readily dissolved in or wetted by non-polar solvents, such as hydrocarbons, than by water). In a particular embodiment, hydrophobic polymers may have aqueous solubility less than about 1% wt. at 37° C. In a particular embodiment, polymers that at 1% solution in bi-distilled water have a cloud point below about 37° C., particularly below about 34° C., may be considered hydrophobic.

As used herein, the term “hydrophilic” means the ability to dissolve in water. In a particular embodiment, polymers that at 1% solution in bi-distilled water have a cloud point above about 37° C., particularly above about 40° C., may be considered hydrophilic.

As used herein, the term “amphiphilic” means the ability to dissolve in both water and lipids/apolar environments. Typically, an amphiphilic compound comprises a hydrophilic portion and a hydrophobic portion.

The term “antimicrobials” as used herein indicates a substance that kills or inhibits the growth of microorganisms such as bacteria, fungi, viruses, or protozoans.

As used herein, the term “antiviral” refers to a substance that destroys a virus and/or suppresses replication (reproduction) of the virus. For example, an antiviral may inhibit and or prevent: production of viral particles, maturation of viral particles, viral attachment, viral uptake into cells, viral assembly, viral release/budding, viral integration, etc.

As used herein, the term “antibiotic” refers to antibacterial agents for use in mammalian, particularly human, therapy. Antibiotics include, without limitation, beta-lactams (e.g., penicillin, ampicillin, oxacillin, cloxacillin, methicillin, and cephalosporin), carbacephems, cephamycins, carbapenems, monobactams, aminoglycosides (e.g., gentamycin, tobramycin), glycopeptides (e.g., vancomycin), quinolones (e.g., ciprofloxacin), moenomycin, tetracyclines, macrolides (e.g., erythromycin), fluoroquinolones, oxazolidinones (e.g., linezolid), lipopetides (e.g., daptomycin), aminocoumarin (e.g., novobiocin), co-trimoxazole (e.g., trimethoprim and sulfamethoxazole), lincosamides (e.g., clindamycin and lincomycin), polypeptides (e.g., colistin), and derivatives thereof.

As used herein, an “anti-inflammatory agent” refers to compounds for the treatment or inhibition of inflammation. Anti-inflammatory agents include, without limitation, non-steroidal anti-inflammatory drugs (NSAIDs; e.g., aspirin, ibuprofen, naproxen, methyl salicylate, diflunisal, indomethacin, sulindac, diclofenac, ketoprofen, ketorolac, carprofen, fenoprofen, mefenamic acid, piroxicam, meloxicam, methotrexate, celecoxib, valdecoxib, parecoxib, etoricoxib, and nimesulide), corticosteroids (e.g., prednisone, betamethasone, budesonide, cortisone, dexamethasone, hydrocortisone, methylprednisolone, prednisolone, tramcinolone, and fluticasone), rapamycin, acetaminophen, glucocorticoids, steroids, beta-agonists, anticholinergic agents, methyl xanthines, gold injections (e.g., sodium aurothiomalate), sulphasalazine, and dapsone.

As used herein, the term “subject” refers to an animal, particularly a mammal, particularly a human.

As used herein, the term “prevent” refers to the prophylactic treatment of a subject who is at risk of developing a condition resulting in a decrease in the probability that the subject will develop the condition.

The term “treat” as used herein refers to any type of treatment that imparts a benefit to a patient afflicted with a disease, including improvement in the condition of the patient (e.g., in one or more symptoms), delay in the progression of the condition, etc.

As used herein, the term “analgesic” refers to an agent that lessens, alleviates, reduces, relieves, or extinguishes pain in an area of a subject's body (i.e., an analgesic has the ability to reduce or eliminate pain and/or the perception of pain).

As used herein, the term “small molecule” refers to a substance or compound that has a relatively low molecular weight (e.g., less than 2,000). Typically, small molecules are organic, but are not proteins, polypeptides, or nucleic acids.

The term “hydrogel” refers to a water-swellable, insoluble polymeric matrix (e.g., hydrophilic polymers) comprising a network of macromolecules, optionally crosslinked, that can absorb water to form a gel.

The term “crosslink” refers to a bond or chain of atoms attached between and linking two different molecules (e.g., polymer chains). The term “crosslinker” refers to a molecule capable of forming a covalent linkage between compounds. A “photocrosslinker” refers to a molecule capable of forming a covalent linkage between compounds after photoinduction (e.g., exposure to electromagnetic radiation in the visible and near-visible range). Crosslinkers are well known in the art (e.g., formaldehyde, paraformaldehyde, acetaldehyde, glutaraldehyde, etc.). The crosslinker may be a bifunctional, trifunctional, or multifunctional crosslinking reagent.

The following example illustrates certain embodiments of the invention. It is not intended to limit the invention in any way.

### EXAMPLE

### Materials and Methods

### Materials

Calcium nitrate tetrahydrate, triethyl phosphate, tetraethyl orthosilicate, polyvinylpyrrolidone (PVP, Mw 1300000), gelatin (type A) from porcine skin, tris(hydroxymethyl)aminomethane, and 3D printed PCL scaffolds were obtained from Sigma-Aldrich (St. Louis, MO). PLGA (50:50 lactide:glycolide) was purchased from Lactel (Birmingham, AL). Collagen type I was obtained from Elastin Products Co., Inc. (Owensville, MO). Acetic acid, sodium acetate trihydrate, and sodium chloride were obtained from Fisher Chemical (Fair Lawn, NJ). Hexafluoroisopropanol and 37% hydrochloric acid were purchased from Oakwood Chemical (Estill, SC). E7-BMP2 (EEEEEEEKIPKASSVPTELSAISTLYL (SEQ ID NO: 1), 3022.28 g/mol) and E7-BMP2-FITC (3524.82 g/mol) peptides were synthesized by Genscript Co., Inc. (Piscataway, NJ). Low glucose DMEM and α-MEM were purchased from Gibco (Grand Island, NY).

### Preparation of PCL PCG and BG Nanofibers

PCG and BG nanofibers were fabricated as described (Weng, et al. (2018) Adv. Healthcare Mater., 7:1701415). Briefly, PCG solution was prepared by dissolving PLGA, gelatin, and collagen in hexafluoroisopropanol at a mass ratio of 2:1:1. PCG nanofibers were electrospun under the conditions shown as follows: 0.4 mL/hour feeding rate, 15 kV applied direct-current voltage, and 15 cm distance between a spinneret and a collecting mandrel at a fast rotation speed. The PCG nanofibers were then cross-linked using glutaraldehyde (GA) vapors for 12 hours. For fabricating BG nanofibers, tetraethyl orthosilicate, triethyl phosphate, and Ca(NO₃)2.4H₂O were dissolved in acidic solution at a [Si]:[P]:[Ca] molar ratio of 80:10:10 and then mixed with an equal volume of 16.5% PVP solution in ethanol. The mixed solution was fed at a rate of 0.6 mL/hour, and the mandrel rotated slowly to collect composite fibers with the same remaining conditions mentioned in the PCG nanofiber preparation. PVP fibers were removed from the composite fibers by sintering in a muffle furnace at 600° C. for 5 hours and BG nanofibers were harvested.

### Preparation of PCL Scaffold-PCG and PCL Scaffold-BG

For fabrication of PCG and BG short nanofibers (SFs), the electrospun PCG and BG fibrous membranes were scissored into small pieces and then cut into fibrous mince by a cryotome, followed by lyophilization. The dry fibrous mince were suspended in acetic buffer (AcB, pH 4.0) at a concentration of 20 mg/mL and homogenized by a 20 kHz probe sonicator (Qsonica 500) equipped with a ⅛ inch probe to generate SFs. The amplitude of sonication was 35% and on/off cycle was 10/10 seconds.

The four-layer PCL meshes were fabricated by melt electrospinning writing (Spraybase, Co. Kildare, Ireland) at 85° C. PCL beads (molecular weight 45,000) were fused in a stainless steel chamber and extruded from a spinneret with a diameter of 0.35 mm under 0.2 bar pressure. The distance between the collector and spinneret was 30 mm, and voltage of 14 kV was applied.

The 3D PCL scaffolds (including meshes) were first treated by plasma under an oxygen atmosphere at 0.4 bar for 20 minutes. For preparing PCL scaffold-BG, plasma-treated PCL scaffolds were immersed in gelatin solution (2 mg/mL in AcB) for 20 min and then transferred into 5 or 10 mg/mL BG SFs suspension. After 1 hour BG SF coating, the scaffolds were immersed in gelatin solution (1 mg/mL in AcB) for 20 minutes and freeze-dried. For fabricating PCL scaffold-PCG, the PCL scaffolds were immersed in gelatin and PCG SF solution (1.5 mg/mL gelatin and 10 or 20 mg/mL PCG SFs in AcB) for 1 hour. Glutaraldehyde vapor from ethanol solution was used to cross-link gelatin and immobilize SFs for 36 hours. The unreacted aldehyde groups were removed through immersing the scaffolds in tris-buffered saline (TBS, pH 7.4) over night and washed with PBS five times. The PCL scaffolds treated by 10 and 20 mg/mL PCG SFs were named as PCL scaffold-PCG10 and PCL scaffold-PCG20, respectively. The PCL scaffolds modified with 5 and 10 mg/mL BG SFs were named as PCL scaffold-BG5 and PCL scaffold-BG10, respectively.

### Analysis of Surface Morphology

The surface morphologies of PCL scaffolds before and after PCG or BG SF modification were determined by an FEI Quanta 200 SEM at a nitrogen atmosphere and an accelerating voltage of 25 kV. Gold and palladium were coated on these scaffolds to avoid charging before acquiring the SEM images.

### Micro-CT Analysis

The morphology of PCL scaffold-BG was also analyzed by a Skyscan1172 micro-CT scanner (Kontick, Belgium) equipped with an X-ray tube. The scanning source voltage and current were 39 kV and 159 μA, respectively, and the image pixel size was set as 2.22 The 3D model of PCL scaffold-BG was reconstructed by CT analyzer software (Bruker micro-CT).

### Mechanical Test

The mechanical properties of PCL scaffold, PCL scaffold-PCG and PCL scaffold-BG were evaluated by compression test using an MTS8800 universal test machine equipped with a 25 kN cell load. The specimens were compressed at a loading rate of 0.002 mm/s to 70% of their original heights.

### In Vitro Cellular Response

The cell attachment and proliferation on PCL scaffold, PCL scaffold-PCG, and PCL scaffold-BG were observed by a Zeiss LSM 800 Airyscan CLSM. MC3T3-E1 cells were seeded on these scaffolds at a density of 2×10⁴ per well in Alpha modified minimum essential medium (α-MEM) supplemented with 10% fetal bovine serum (FBS) and 1% penicillin/streptomycin in a 96-well plate and the CLSM images of attachment were collected after 2, 4, and 12 hour culture. MC3T3-E1 cell proliferation on these scaffolds was determined after 4 day culture. The effect of these scaffolds on cell attachment and proliferation of rBMSC in low glucose Dulbecco's modified eagle medium (DMEM) with 10% FBS and 1% penicillin/streptomycin (growth medium) were also detected after 12 hour and 7 day culture. The seeding density of rBMSC was 2×10⁴ in each well. Cells on these scaffolds were fixed by 4% paraformaldehyde in phosphate-buffered saline (PBS) overnight. Then, the cells were washed with PBS twice. F-actin and nucleus were stained with Alexa Fluor® 546 phalloidin and 4′,6-diamidino-2-phenylindole (DAPI), respectively, according to manufacturer's manuals. Cell counts were conducted by summing the cell numbers in a region acquired by CLSM. The final cell number is the average of counts in three different regions randomly chosen.

SEM was used to further confirm the influence of PCG and BG SF decoration on cell attachment and proliferation. The cells were fixed with solution containing 2% paraformaldehyde and 2% glutaraldehyde and dehydrated with 10%, 20%, 30%, 50%, 75%, and 100% ethanol solutions in turn. The SEM images were captured at an accelerating voltage of 25 kV after the samples were coated with gold and palladium by a sputter coater.

Loading and Release of E7-BMP2 Peptide

BMP2 peptide was introduced by immersing PCL scaffold-BG in E7-BMP2 peptide or E7-BMP2-FITC solution (100 or 300 μg/mL in TBS) at room temperature for 12 or 24 hours. After washed with PBS three times and freeze-dried, the obtained PCL scaffold-BG-BMP2 and PCL scaffold-BG-BMP2-FITC were stored at −20° C. The difference between PCL scaffold-BG and PCL scaffold-BG-BMP2-FITC was distinguished using a Zeiss LSM 800 Airyscan CLSM at an excitation wavelength of 488 nm. The concentration of released peptide from PCL scaffold-BG-BMP2-FITC was periodically recorded by a Synergy H1 hybrid reader which measured the fluorescent intensity of E7-BMP2-FITC at the excitation and emission wavelengths of 485 and 528 nm, respectively.

rBMSC Osteogenic Differentiation on BMP2-Loaded PCL Scaffold-BG

A PCL scaffold as a control, PCL scaffold-BG, and PCL scaffold-BG-BMP2 were immersed in growth medium with 2×10⁴ rBMSCs (passage 3 or 4) in a 96-well plate for 2 days. Then, the growth medium was substituted with OsteoMAX-XF differentiation medium (ODM) to culture the cells for 7 days. The ODM was changed every 3 days. RNeasy® mini kit (Qiagene) and cDNA synthesis kit (Quanta) were used to extract and reverse transcribe the total RNA, respectively. The relative mRNA expression levels of Runt-related transcription factor 2 (Runx2), alkaline phosphatase (Alp), osteocalcin (OCN) and bone sialoprotein (BSP), which are osteogenic makers, were quantified by using SYBR®-Green qRT-PCR kit. The expression of individual gene was calculated relative to the GAPDH expression. The primers are listed in Table 1.

TABLE 1
Primers. SEQ ID Nos are provided in parentheses.

| | Forward primer | Reverse primer |
|---|---|---|
| Gene | (SEQ ID NO) | (SEQ ID NO) |
| Runx2 | CCAACTTCCTGTGCTCCGTG (2) | GTGAAACTCTTGCCTCGTCCG (3) |
| Alp | CGGCCATCCTATATGGTAACGG (4) | CAGGAGGCATACGCCATCACA (5) |
| OCN | GACCCTCTCTCTGCTCACTCT (6) | GACCTTACTGCCCTCCTGCTTG (7) |
| BSP | AGGTCGTCAACGGCACCAGC (8) | CCATAGGCTTCGGGTGGCGG (9) |
| GAPDH | GCAAGTTCAACGGCACAG (10) | CGCCAGTAGACTCCACGAC (11) |

### Statistical Analysis

Each experiment was performed at least three replicates. The data was analyzed using the SPSS software. The statistical analysis was performed using one-way analysis of variance with Tukey test, and p*<0.05 indicated a statistical significance.

### Results

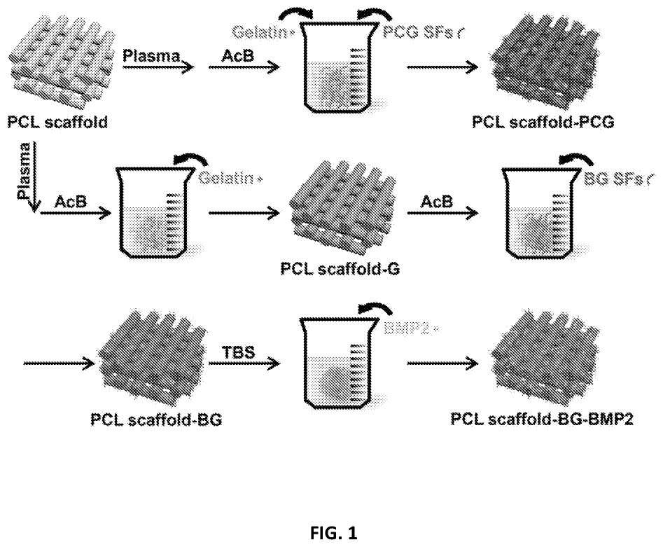

FIG. 1 shows the schematic illustrating the fabrication process of poly(ε-caprolactone) (PCL) scaffold-PCG and PCL scaffold-BG. The PCL scaffolds were first treated with plasma under an oxygen atmosphere. Plasma treatment creates radicals at the midpoint of PCL chains via abstracting hydrogen, and the excited radicals from plasma oxygen react with those on polymers to form oxygen-containing functionalities such as hydroxy and carboxyl groups, thus making the PCL scaffolds hydrophilic (Chua, et al. (2002) Mater. Sci. Eng. R Rep., 36:143; Pappa, et al. (2015) J. Nanotechnol., 6:254). The generated negatively charged carboxyl groups enhance the electrostatic interactions between the PCL scaffolds and gelatin as well as PCG short nanofibers (SFs) (diameter=˜235 nm, length=2-8 μm) in acetic buffer (AcB) (pH 4.0), wherein gelatin exhibits a positive charge within this pH range (Djagny, et al. (2001) Crit. Rev. Food Sci. Nutr., 41:481). 3D printed PCL scaffolds were coated with PCG SFs by immersing the scaffolds in AcB containing 1.5 mg/mL of gelatin and 10 mg/mL or 20 mg/mL of PCG SFs for 1 hour. For coating with BG SFs (diameter=˜382 nm, length=6-13 μm), PCL scaffolds were treated with 2 mg/mL of gelatin solution in AcB, and 5 mg/mL or 10 mg/mL of BG SFs suspension in AcB was used to replace the gelatin solution, followed by transferring the scaffolds to a gelatin solution at a lower concentration (1 mg/mL). Glutaraldehyde was used to cross-link gelatin to prevent its dissolution in medium and SFs falling off the scaffolds. Here, gelatin served as an adhesive to build up ECM-mimicking SF architecture on the surface of plasma-treated 3D printed scaffolds.

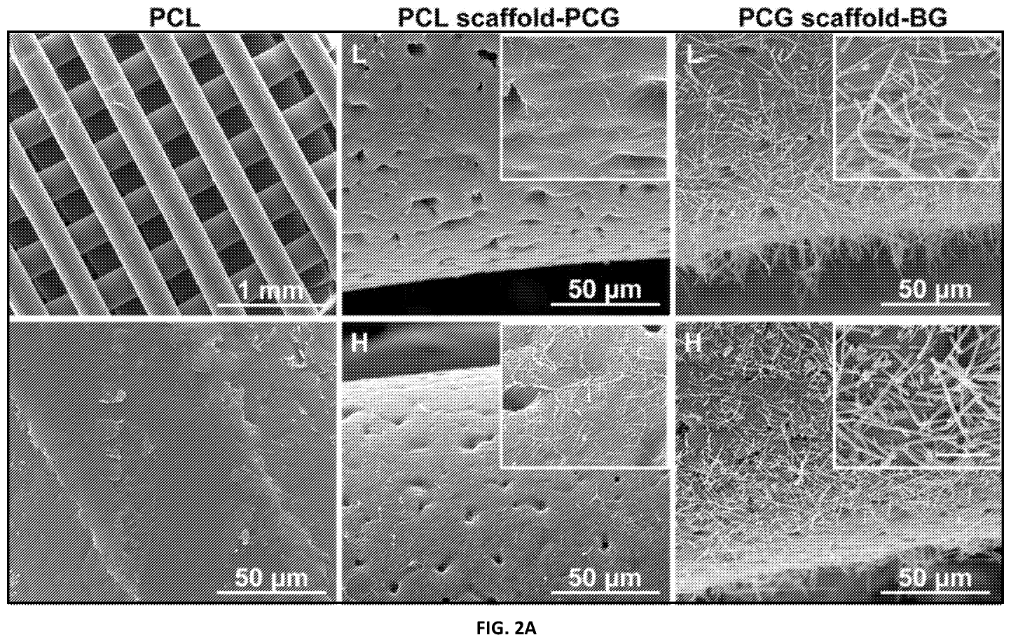

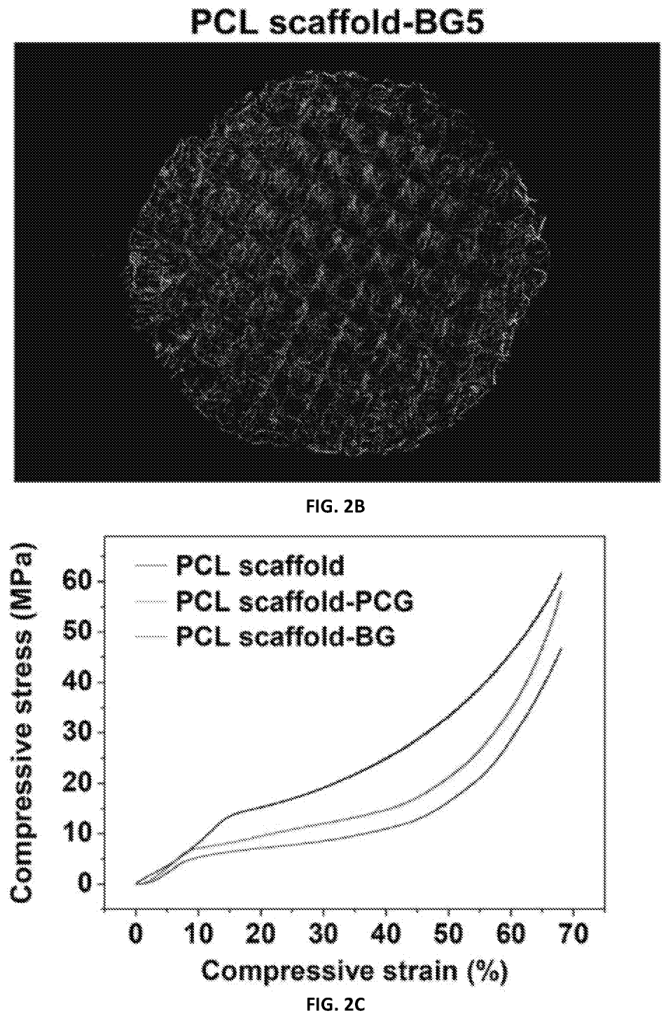

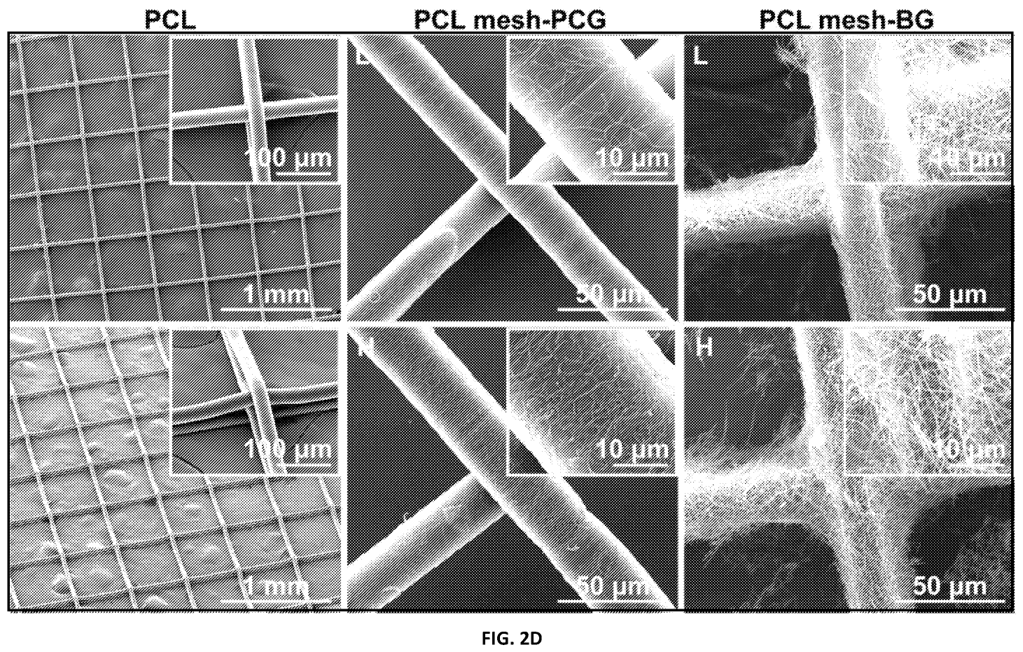

FIG. 2A shows scanning electron microscopy (SEM) images of 3D printed PCL scaffolds made up of orthogonal microfibers with a diameter of 291.6 μm, displaying 300 μm-sized interconnected pores. These pores can facilitate deep cell infiltration into the scaffolds and enable nutrient transport. The density of SFs adherent on the surface of PCL scaffolds may be controlled via adjusting the concentration of SFs in the suspension. As PCG SF and BG SF concentrations increased, the surface coverage of coated SFs on PCL scaffolds also increased. Coating with PCG and BG SFs significantly changed the surface topography of the PCL scaffolds. The gelatin concentration played a key role in the immobilization of SFs. Solutions with a low gelatin concentration resulted in poor SFs attachment, while solutions with a high gelatin concentration led to a thicker gelatin coating layer which partially or completely covered the surface-attached SFs, likely blocking the interactions between cells and SFs. However, the decorated BG SFs tended to stack up, leading to insufficient attachment for some BG SFs and release from the scaffold, which may compromise cell adhesion. The extra gelatin coating to PCL scaffold-BG was necessary for further immobilization through enhancing joints between BG SFs. The aforementioned surface-decorating method was also used to modify PCL meshes generated by melt electrospinning writing (FIG. 2D). The four-layered PCL meshes consisted of microfibers with a diameter of 20-30 μm. Different densities of PCG SFs and BG SFs were grafted onto these PCL meshes by controlling the SF concentrations in the corresponding suspensions. To further confirm the BG SF modification on PCL scaffolds, microcomputed tomography (micro-CT) was applied to quantitatively analyze the coating efficiency. FIG. 2B shows the homogeneous distribution of BG SFs on the surface of a PCL scaffold. For PCL scaffold-BG5 (PCL scaffold treated by 5 mg/mL of BG SFs suspension), the percentage of the surface area covered by BG SFs to the total surface area was 53.8%, further validating the coating method in decorating 3D printed scaffolds with SFs.

To investigate the effect of nanofiber segment decoration on mechanical properties, compression tests were performed on PCL scaffolds, PCL scaffold-PCG, and PCL scaffold-BG. FIG. 2C shows the mechanical properties of PCL scaffolds and SF-modified PCL scaffolds. There were three regions in these compressive strain curves. In the first region where the compressive strain was less than 10%, the moduli of PCL scaffold, PCL scaffold-PCG, and PCL scaffold-BG were similar probably because the pores were compressed at the beginning. The difference in compressive stresses between PCL scaffolds, PCL scaffold-PCG, and PCL scaffold-BG became larger as the compressive strain increased from 10% to 45% (the second region). The possible reason was that the soft gelatin layers and PCG SFs and the fragile, loose-stacked BG SFs provided some buffer room, bearing some load before the bulk load was transferred to the scaffolds. When the deformation reached above 45% (the third region), the stresses of SF-modified scaffolds became closer to that of PCL scaffolds, indicating that the PCL substrate played a more important role in the mechanical property compared to the decorated architectural surfaces. In addition, the PCG SFs and BG SFs decorations increased the strut size by 13.1 and 17.2 respectively, (compared to PCL scaffold ˜290 μm) and the porosities correspondingly decreased by 3.5% and 4.6%, respectively, compared to porosity of PCL scaffold (62.4%), indicating this surface modification of nanotopology had no significant effect on the micro- and macro-architectures.

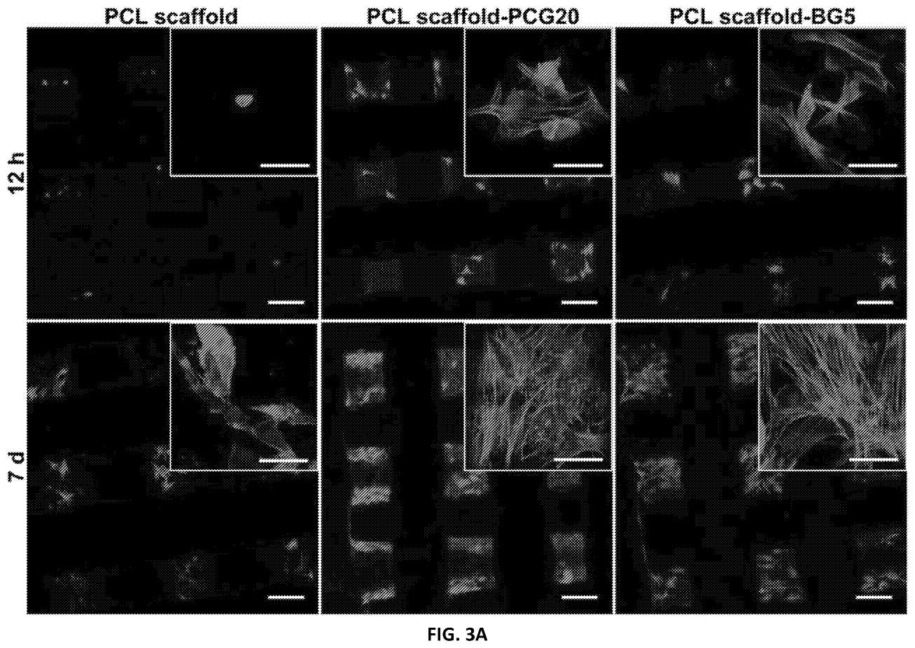

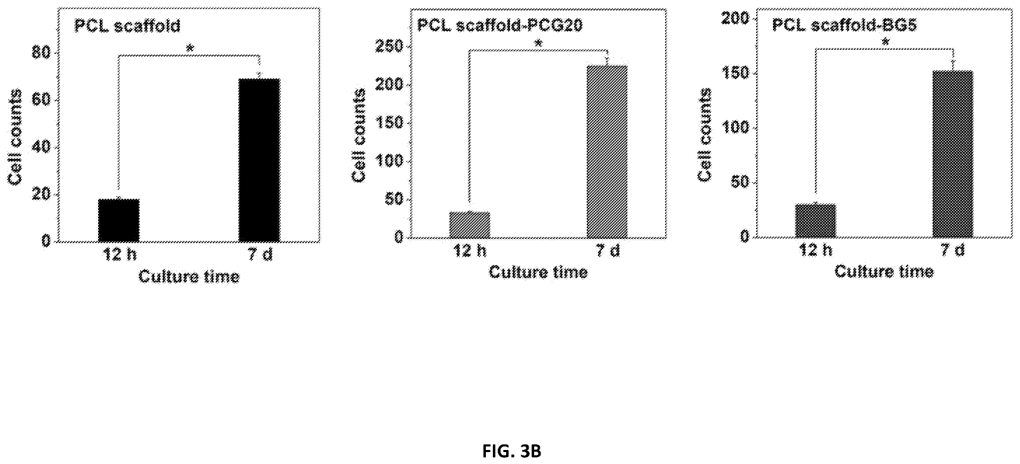

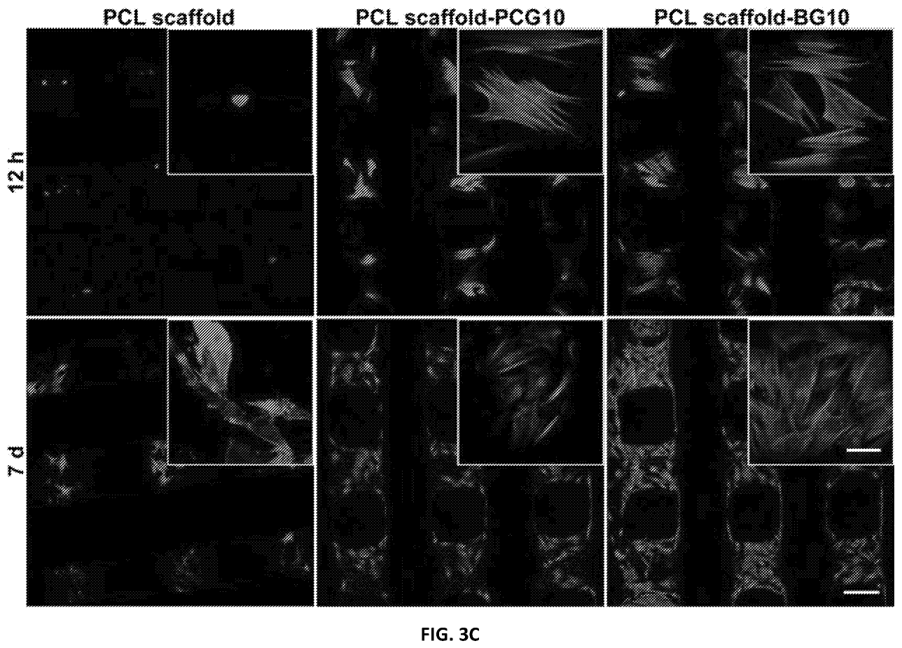

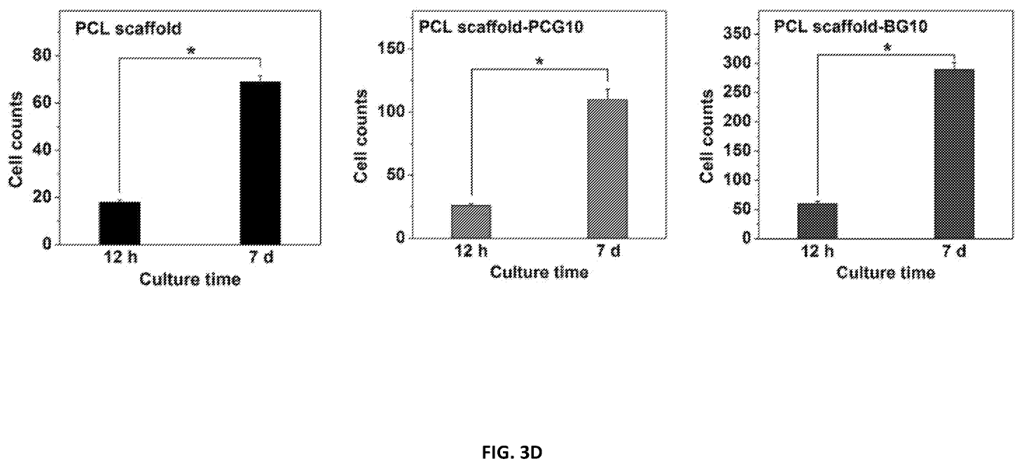

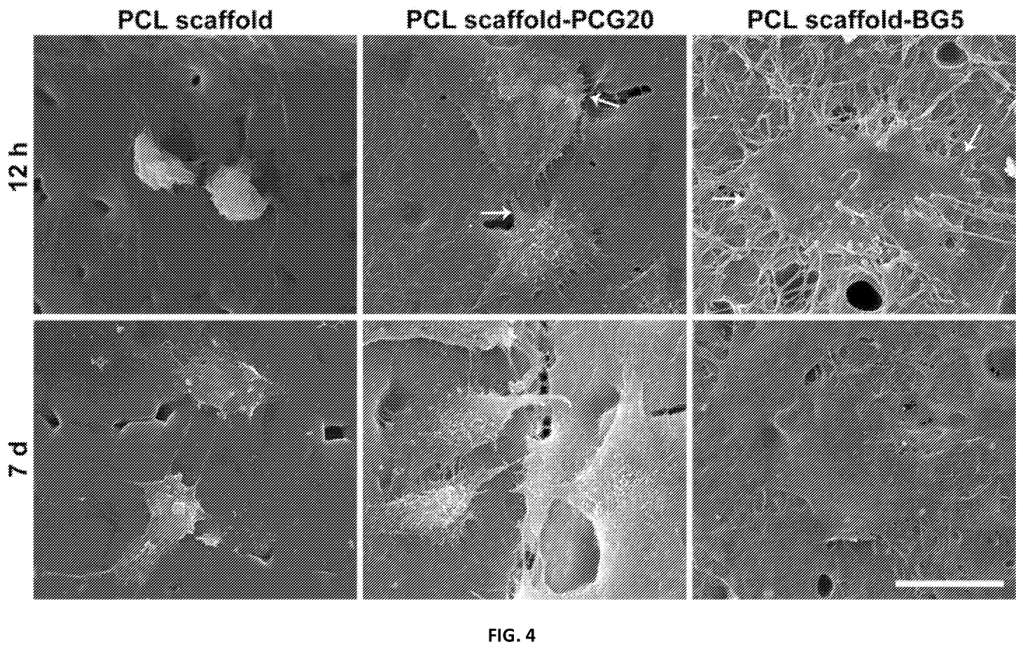

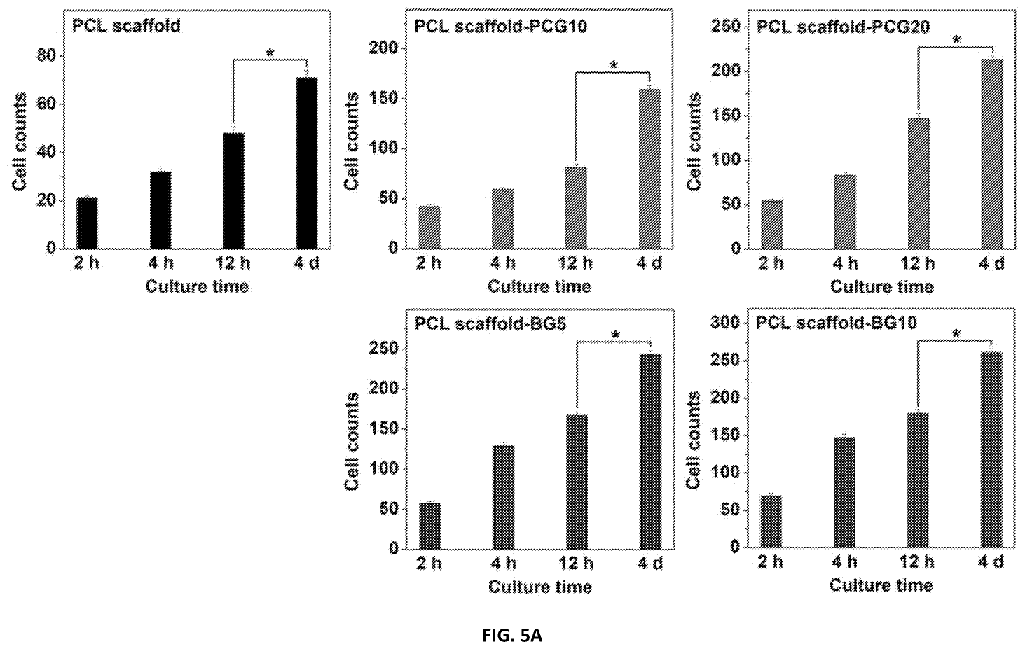

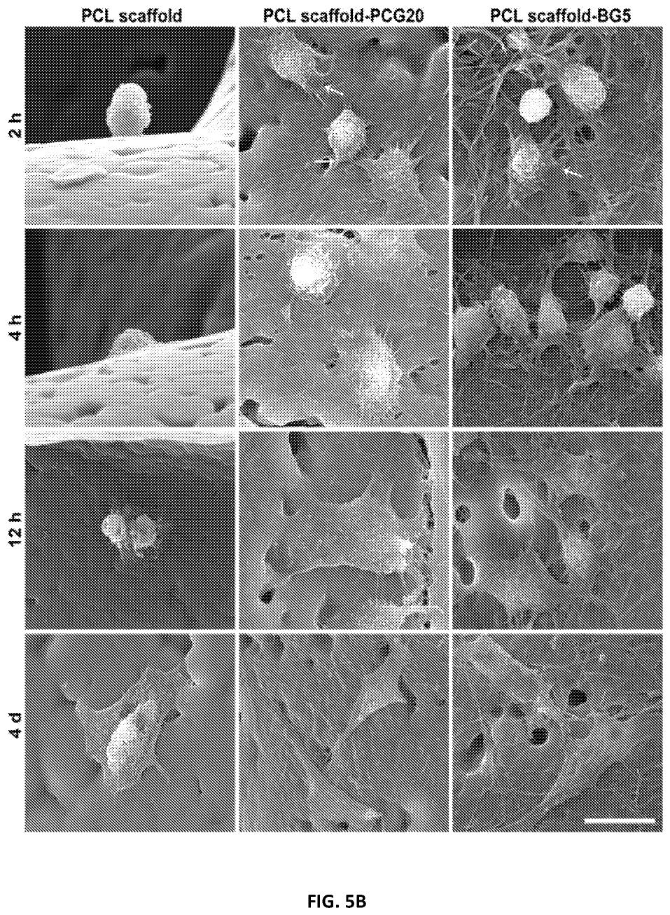

To examine the effect of nanofiber segment decoration on cellular responses, rat bone marrow-derived stem cells (rBMSCs) were seeded onto PCL scaffolds, PCL scaffolds-PCG, and PCL scaffolds-BG, and cell adhesion and proliferation over different culture periods were examined. FIG. 3A shows confocal laser scanning microscopy (CLSM) images illustrating the better cell adhesion on PCL scaffolds-PCG20 and PCL scaffolds-BG5 than on PCL scaffolds. Similarly, PCL scaffold-PCG10 and PCL scaffold-BG10 were more favorable to cell attachment than PCL scaffolds (FIG. 3C). The insets also indicate that BMSCs exhibited much better spreading and evident F-actin bundles and stress fibers on the PCL scaffold-PCG and PCL scaffold-BG than those on the PCL scaffolds (FIGS. 3A and 3C). The profiles of cell numbers for various scaffolds demonstrated that the adhesion rates of rBMSC on the SF-decorated scaffolds were approximately twice as high as that on PCL scaffolds after seeding for 12 hours (FIG. 3B). In addition, rBMSCs grew faster and distributed more extensively on the PCL scaffold-PCG20 and PCL scaffold-BG5 after 7-day culture. The density of decorated SFs on the scaffold affected cellular responses. The cell numbers were higher on PCL scaffold-PCG20 (or PCL scaffold-BG10) than on PCL scaffold-PCG10 (or PCL scaffold-BG5) over 12 hours and 7 days of incubation, indicating the increased SF density contributed to cell adhesion and proliferation (FIGS. 3B and 3D). One of the reasons could be that the decoration with high density of SFs formed the nanoarchitecture closer to structure of ECM. As shown in FIG. 4, rBMSCs were loosely attached and maintained the spherical shape on the PCL scaffold at 12 hours, and such cellular behavior was significantly different from that on the SF-modified scaffolds. Better cell adhesion and spreading occurred on PCL scaffold-PCG20 and PCL scaffold-BG5. More rBMSCs were observed on SF-decorated surfaces than on the untreated surface after 7-day culture. The results of SEM were consistent with those of CLSM. MC3T3-E1 pre-osteoblasts were also seeded to reaffirm the cellular responses to decorations of PCG SFs and BG SFs on the 3D printed PCL scaffolds at different time points. CLSM images showed the poor adhesion of MC3T3-E1 cells on the PCL scaffolds at 2 hours, 4 hours, and even 12 hours. The number of cells attached on the PCL scaffolds increased slowly over time (FIG. 5A). In contrast, more cells were found on the PCL scaffold-PCG and PCL scaffold-BG at 2 hours. PCG SFs and BG SFs made the scaffolds much more appealing to MC3T3-E1 cells, and much larger surface areas were occupied by cells on PCL scaffold-PCG and PCL scaffold-BG than those on unmodified PCL scaffolds at 4 hours and 12 hours. Furthermore, the decoration of PCG SFs and BG SFs significantly promoted MC3T3-E1 cell proliferation compared with PCL scaffolds over a long culture period (4 days). The higher number of cells for attachment and proliferation were due to the increase of the SF density on the surface (FIG. 5A).

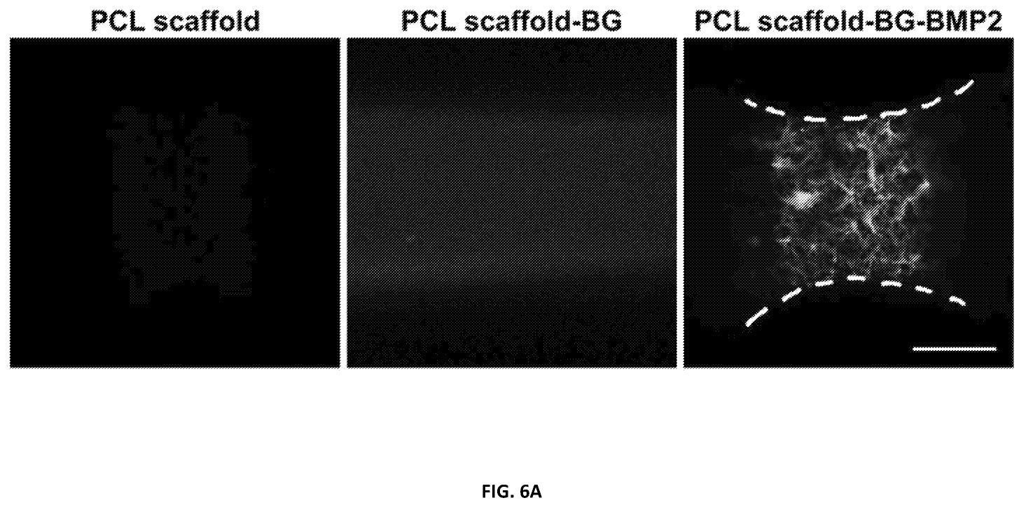

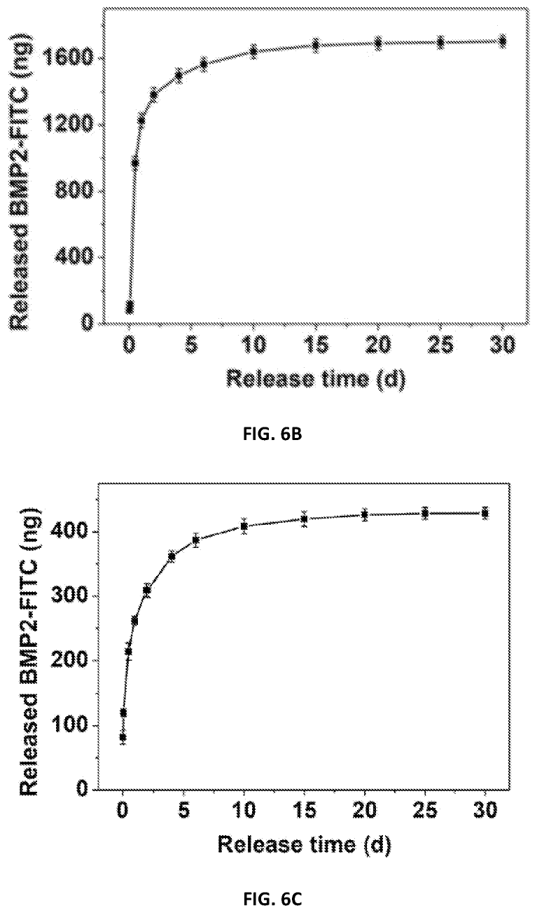

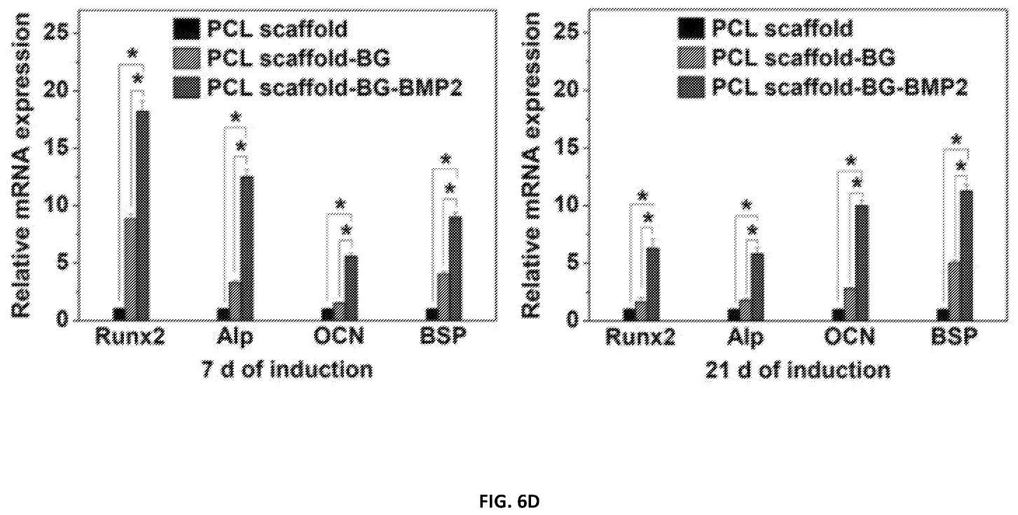

The SEM images in FIG. 5B confirmed that MC3T3-E1 cells proliferated poorly on PCL scaffold over short culture periods (2 hours to 12 hours). Adherent cells demonstrated an ellipsoidal morphology at 2 hours and gradually spread as the culture time increased to 12 hours, but still had no obvious protrusions. Though the cells on PCL scaffold-PCG20 and PCL scaffold-BG5 did not spread thoroughly at 2 hours, pseudopods were clearly observed. The spreading areas in the PCG20 and BG5 groups increased quickly when the culture time reached 12 hours, which were much larger than those on PCL scaffold. MC3T3-E1 cells on all of the scaffolds spread thoroughly at 4 days, indicating that the poorly attached cells at the beginning were able to spread and proliferate after culturing for a sufficient time. These results indicated that the decoration of PCG SFs and BG SFs improved the attachment, spreading, and proliferation of rBMSCs and pre-osteoblasts on 3D printed PCL scaffolds. To further enhance the regenerative capabilities of the 3D printed constructs, E7-BMP2 peptide was conjugated to the BG SF-modified PCL scaffolds to enable osteogenic differentiation-inducing properties. The modification with BMP2 was achieved through surface coupling the heptaglutamate domain (E7) of the peptide to the Ca on BG SFs on the PCL scaffolds (Culpepper, et al. (2010) Biomaterials 31:9586). E7-BMP2-fluorescein isothiocyanate (FITC) was used to detect the loading and release of the peptide. As shown in FIG. 6A, no fluorescence was detected on the PCL scaffold. The weak green fluorescence was observed on the PCL scaffold-BG, which could be due to the autofluorescence of the gelatin coating. In contrast, E7-BMP2-FITC modified PCL scaffold-BG exhibited intense green fluorescence, indicating the presence of E7-BMP2 peptides. The PCL scaffold-BG-BMP2 released BMP2 over a 30 day period, marked by an intial burst release due to physically absorbed BMP2-FITC, and a steady release after day 2 (FIG. 6B). The gradual BMP2 release was attributed to E7 domain which modeled the process that a number of bone-matrix proteins binded to the calcium of bone because they possessed stretches of negatively-charged amino acids (Sawyer, et al. (2005) Biomaterials 26:7046; Hoang, et al. (2003) Nature 425:977). The total amount of incorporated BMP2 peptides could be adjusted by changing the initial peptide concentration and the coupling time. When the concentration and coupling time decreased from 300 to 100 μg/mL and 24 to 12 hours, respectively, the amount of released peptide was changed from 1704 ng to 429 ng per scaffold at 30 days (FIG. 6C).

To examine the effect of the incorporated BMP2 peptides on cellular response, the PCL scaffold-BG treated with a higher concentration of E7-BMP2 peptide was used to test osteogenic markers of BMSCs. The relative mRNA expression levels of a series of osteogenic markers in rBMSC were quantified by qRT-PCR after rBMSCs were cultured on PCL scaffolds, PCL scaffolds-BG, and PCL scaffolds-BG-BMP2 in osteogenic differentiation medium for 7 days and 21 days (FIG. 6D). Compared with the untreated PCL scaffolds, the BG SF-decorated PCL scaffolds demonstrated significantly increased stimulation of rBMSCs towards osteogenesis. Moreover, mRNA expression levels of Runx2, Alp, OCN, and BSP were significantly higher in BMSCs cultured on PCL scaffolds-BG-BMP2 compared to the other two scaffolds. Therefore, the osteogenic differentiation was further improved in response to the BMP-2 peptides immobilized on PCL scaffolds-BG. In addition, OCN and BSP, which are late marker genes for osteogenesis, exhibited an increase in expression levels with increasing the induction time from 7 days to 21 days, while the expression levels of early-stage osteogenic markers Runx2 and Alp decreased with the progression of rBMSCs differentiation on PCL scaffold-BG-BMP2.

In conclusion, a simple and versatile method to decorate scaffolds (e.g., 3D printed scaffolds) with nanofibers (e.g., electrospun nanofiber segments) and bioactive materials is provided. The method has unique features, including broad selection over compositions of nanofiber segments, versatility to incorporate bioactive materials, no compromise of mechanical properties of 3D printed scaffolds, and maintenance of macroscale porosity. The PCG SF- and BG SF-decorated PCL scaffolds showed improved cell adhesion and proliferation for rBMSCs and MC3T3-E1 cells. Introducing BMP2 peptides further allowed the SF-modified 3D scaffolds to exhibit pronounced differentiation-inducing properties. The combination of 3D printing and electrospinning techniques results in fabrication of functional scaffolds for tissue engineering.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. A method of synthesizing a surface modified scaffold, said method comprising coating the surface of the scaffold with a coating material and nanofiber segments, and crosslinking the coating on the surface of the scaffold, thereby generating the surface modified scaffold.

2. The method of claim 1, wherein said method comprises plasma treating the scaffold prior to coating the surface of said scaffold.

3. The method of claim 1, wherein said crosslinking comprises contacting the coated scaffold with a chemical crosslinker.

4. The method of claim 3, wherein said chemical crosslinker is glutaraldehyde.

5. The method of claim 1, further comprising synthesizing the scaffold prior to coating.

6. The method of claim 5, wherein said scaffold is synthesized by 3D printing.

7. The method of claim 1, wherein said nanofiber segments have a length less than about 50 μm.

8. The method of claim 1, wherein said method further comprises adding an agent to the coating of said surface modified scaffold.

9. The method of claim 1, wherein said coating material is gelatin.

10. The method of claim 1, wherein said nanofiber segments are electrospun nanofiber segments.

11. The method of claim 1, wherein said scaffold is coated with the coating material by immersing or soaking the scaffold in a solution or suspension comprising the coating material and, optionally, the nanofiber segments and/or an agent.

12. The method of claim 1, wherein said scaffold comprises microfibers comprising polycaprolactone.

13. The method of claim 1, further comprising adding cells, tissues, and/or an agent to the surface modified scaffold.

14. The method of claim 8, wherein said agent is selected from the group consisting of a therapeutic agent, a growth factor, a signaling molecule, a cytokine, a hemostatic agent, an antimicrobial, and an antibiotic.

15. The surface modified scaffold synthesized by the method of claim 1.

16. A surface modified scaffold comprising a) a plasma treated scaffold and b) a crosslinked coating comprising a coating material and nanofiber segments.

17. The surface modified scaffold of claim 16, wherein said scaffold is a 3D printed scaffold.

18. The surface modified scaffold of claim 16, wherein said nanofiber segments have a length less than about 50 μm.

19. The surface modified scaffold of claim 16, wherein said coating material is gelatin.

20. The surface modified scaffold of claim 16, wherein said nanofiber segments are electrospun nanofiber segments.

21. The surface modified scaffold of claim 16, wherein said scaffold comprises microfibers comprising polycaprolactone.

22. The surface modified scaffold of claim 16, wherein said surface modified scaffold further comprises an agent, cells, and/or tissues in the coating.

23. The surface modified scaffold of claim 22, wherein said agent is selected from the group consisting of a therapeutic agent, a growth factor, a signaling molecule, a cytokine, a hemostatic agent, an antimicrobial, and an antibiotic.
